SEQUENCE LISTING (45 sequences)

SEQ ID NO: 1 - protein, 357 aa, synthetic construct
  REGION 1..357: note = Synthetic Sequence
DVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 2 - protein, 357 aa, synthetic construct
  REGION 1..357: note = Synthetic Sequence
DVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3 - protein, 357 aa, synthetic construct
  REGION 1..357: note = Synthetic Sequence
DVQLVESGGGLVQPGGSLRLSCAASGRIFSVNAMGWYRQAPGKQRELVAAITNQGAPTYADSVKGRFTISRDNSKNTVYLQMNSLRPEDTAVYYCKAFTRGDDYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 4 - protein, 357 aa, synthetic construct
  REGION 1..357: note = Synthetic Sequence
DVQLVESGGGLVQPGGSLRLSCAASGRIFSVNAMGWYRQAPGKQRELVAAITNQGAPTYADSVKGRFTISRDNSKNTVYLQMNSLRPEDTAVYYCKAFTRGDDYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 5 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 6 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIMASFSLSTNLQESLRSKE

SEQ ID NO: 7 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 8 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIMASFSLSTNLQESLRSKE

SEQ ID NO: 9 - protein, 165 aa, synthetic construct
  REGION 1..165: note = Synthetic Sequence
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 10 - protein, 164 aa, synthetic construct
  REGION 1..164: note = Synthetic Sequence
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEWRAEIMRSFSLSTNLQESLRSKE

SEQ ID NO: 11 - protein, 115 aa, synthetic construct
  REGION 1..115: note = Synthetic Sequence
DVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSS

SEQ ID NO: 12 - protein, 115 aa, synthetic construct
  REGION 1..115: note = Synthetic Sequence
DVQLVESGGGLVQPGGSLRLSCAASGRIFSVNAMGWYRQAPGKQRELVAAITNQGAPTYADSVKGRFTISRDNSKNTVYLQMNSLRPEDTAVYYCKAFTRGDDYWGQGTLVTVSS

SEQ ID NO: 13 - protein, 227 aa, synthetic construct
  REGION 1..227: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 14 - protein, 227 aa, synthetic construct
  REGION 1..227: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 227 aa, synthetic construct
  REGION 1..227: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 16 - protein, 227 aa, synthetic construct
  REGION 1..227: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 487 aa, synthetic construct
  REGION 1..487: note = Synthetic Sequence
DVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 18 - protein, 487 aa, synthetic construct
  REGION 1..487: note = Synthetic Sequence
DVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGSGGSGGSGGSGGSDVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSS

SEQ ID NO: 19 - protein, 553 aa, synthetic construct
  REGION 1..553: note = Synthetic Sequence
DVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 20 - protein, 487 aa, synthetic construct
  REGION 1..487: note = Synthetic Sequence
QVQLQESGGGLVQAGGSLRLSCTASGTIFSINRMDWFRQAPGKQRELVALITSDGTPAYADSAKGRFTISRDNTKKTVSLQMNSLKPEDTAVYYCHVSSGVYNYWGQGTQVTVSSGGSGGSGGSGGSGGSDVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 487 aa, synthetic construct
  REGION 1..487: note = Synthetic Sequence
DVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGSGGSGGSGGSGGSQVQLQESGGGLVQAGGSLRLSCTASGTIFSINRMDWFRQAPGKQRELVALITSDGTPAYADSAKGRFTISRDNTKKTVSLQMNSLKPEDTAVYYCHVSSGVYNYWGQGTQVTVSS

SEQ ID NO: 22 - protein, 553 aa, synthetic construct
  REGION 1..553: note = Synthetic Sequence
QVQLQESGGGLVQAGGSLRLSCTASGTIFSINRMDWFRQAPGKQRELVALITSDGTPAYADSAKGRFTISRDNTKKTVSLQMNSLKPEDTAVYYCHVSSGVYNYWGQGTQVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 23 - protein, 647 aa, synthetic construct
  REGION 1..647: note = Synthetic Sequence
MKLPVRLLVLMFWIPASSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSQVQLQESGGGLVQPGGSLRLSCAASGRIFSVNAMGWYRQAPGKQRELVAAITNQGAPTYADSVKGRFTISRDNAGNTVYLQMNSLRPEDTAVYYCKAFTRGDDYWGQGTQVTVSSGGSGGSGGSGGSGGSGGSGGSGGSGGSGGSGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 24 - protein, 647 aa, synthetic construct
  REGION 1..647: note = Synthetic Sequence
MGWSCIIFFLVATATGVHSQVQLQESGGGLVQPGGSLRLSCAASGRIFSVNAMGWYRQAPGKQRELVAAITNQGAPTYADSVKGRFTISRDNAGNTVYLQMNSLRPEDTAVYYCKAFTRGDDYWGQGTQVTVSSGGSGGSGGSGGSGGSGGSGGSGGSGGSGGSGGSGGSGGSGGSGGSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 25 - protein, 353 aa, synthetic construct
  REGION 1..353: note = Synthetic Sequence
QVQLQESGGGLVQPGGSLRLSCAASGRIFSVNAMGWYRQAPGKQRELVAAITNQGAPTYADSVKGRFTISRDNAGNTVYLQMNSLRPEDTAVYYCKAFTRGDDYWGQGTQVTVSSVDGGSGGSGGSGGSGGSGGSRSGGSGGSGGSGGSGGSGGSGGSGGSGGSGGSGGSGGSGGSAAAMCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKELEHHHHHH

SEQ ID NO: 26 - protein, 402 aa, synthetic construct
  REGION 1..402: note = Synthetic Sequence
QVQLQESGGGLVHPGGSLRLSCAASGSFSSINVMGWYRQAPGKERELVARITNLGLPNYADSVTGRFTISRDNAKNTVYLQMNSLKPEDTAVYYCYLVALKAEYWGQGTQVTVSSGGSGGSGGSGGSGGSGGSGGSGGSGGSGGSGGSGGSGGSGGSGGSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 27 - protein, 452 aa, synthetic construct
  REGION 1..452: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRGEIMRSFSLSTNLQESLRSKE

SEQ ID NO: 28 - protein, 452 aa, synthetic construct
  REGION 1..452: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIARSFSLSTNLQESLRSKE

SEQ ID NO: 29 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIARSFSLSTNLQESLRSKE

SEQ ID NO: 30 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIARSFSLSTNLQESLRSKE

SEQ ID NO: 31 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRGEIMRSFSLSTNLQESLRSKE

SEQ ID NO: 32 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRGEIMRSFSLSTNLQESLRSKE

SEQ ID NO: 33 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIARSFSLSTNLQESLRSKE

SEQ ID NO: 34 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIARSFSLSTNLQESLRSKE

SEQ ID NO: 35 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRGEIMRSFSLSTNLQESLRSKE

SEQ ID NO: 36 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRGEIMRSFSLSTNLQESLRSKE

SEQ ID NO: 37 - skipped

SEQ ID NO: 38 - protein, 227 aa, synthetic construct
  REGION 1..227: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 39-40 - skipped (2 entries)

SEQ ID NO: 41 - protein, 422 aa, synthetic construct
  REGION 1..422: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGSGGSGGSGGSGGSGGSGGSGGSGGSGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRGEIMRSFSLSTNLQESLRSKE

SEQ ID NO: 42 - protein, 422 aa, synthetic construct
  REGION 1..422: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRGEIMRSFSLSTNLQESLRSKE

SEQ ID NO: 43 - protein, 356 aa, synthetic construct
  REGION 1..356: note = Synthetic Sequence
DVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 44 - protein, 376 aa, synthetic construct
MEFGLSWLFLVAILKGVQCDVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 45 - protein, 445 aa, synthetic construct
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

(19) United States
(12) Patent Application Publication
(10) Pub. No.: US 2026/0335411 A1
(43) Pub. Date: Oct. 29, 2026

(54) CLEC9A-BASED CHIMERIC PROTEIN FORMULATIONS

(71) Applicant: Orionis Biosciences, Inc., Waltham, MA (US)

(72) Inventors: Nikolai KLEY, Waltham, MA (US); Erik DEPLA, Zwijnaarde (BE); Dorien VAN LYSEBETTEN, Diepenbeek (BE)

(21) Appl. No.: 19/491,093

(22) PCT Filed: Jun. 7, 2024
(86) PCT No.: PCT/US2024/032931

§ 371 Date: Dec. 8, 2025

Related U.S. Application Data
(60) Provisional application No. 63/507,263, filed on Jun. 9, 2023.

(51) Int. Cl. A61K 38/21; A61K 38/17; A61K 47/02; A61K 47/20; A61K 47/22

(52) U.S. Cl. CPC A61K 38/212; A61K 38/1732; A61K 47/02; A61K 47/20; A61K 47/22

(57) ABSTRACT

The present application provides, in part, pharmaceutical formulations of Clec9A-based chimeric protein complexes, and uses thereof.

### CROSS-REFERENCE TO RELATED APPLICATIONS

This application claims the benefit of U.S. Provisional Patent Application No. 63/507,263, filed Jun. 9, 2023, the entire contents of which are hereby incorporated by reference in their entirety.

### FIELD

The present application provides, in part, pharmaceutical formulations of Clec9A-based chimeric protein complexes, and uses thereof.

### SEQUENCE LISTING

This application contains a Sequence Listing in XML format submitted electronically herewith via Patent Center. The contents of the XML copy, created on Jun. 6, 2024, is named “ORN-091PC_114384-5091.xml” and is 68,995 bytes in size. The Sequence Listing is incorporated herein by reference in its entirety.

### BACKGROUND

Biologics with an effector function are a class of biologics that have many potential therapeutic applications. In some instances, these biologics, e.g., cytokines, encode an effector functions that can be systemically toxic if administered to humans. Accordingly, maximizing tolerability and therapeutic index of these biologics in humans is important so that systemic toxicity in humans or subjects can be reduced.

Often, these biologics need be delivered to their target(s) inside a subject with high precision and in a regulated manner in order for them to be effective. Thus, there is a need for engineering biological molecules that have high inherent safety profile, have the ability to reach their target inside the subject with high precision, and are able to function in a regulated fashion.

One example of such biologics, is a chimeric protein having a signaling agent (having an effector function, e.g., a cytokine), connected to a targeting element (having the ability to seek its target with high precision). In these biologics, the signaling agent can be a wild type signaling agent or a modified signaling agent (e.g. by mutation). The modified signaling agent is, generally, modified to cause an attenuation of the signaling agent's activity (e.g., substantially reducing its ability to interact with/engage its receptor) in a manner such that the signaling agent's effector function can be recovered upon binding of the targeting element to its target (e.g., antigen on target cell).

However, such chimeric proteins are amenable to therapeutic use only if certain conditions are met, e.g., the ability to be produced in a large scale, an in vivo half-life that ensures adequate time of exposure to the drug to elicit a therapeutically beneficial effect, a proper size to avoid rapid clearance or limited tissue penetrance and bio-distribution, and other properties that ensure adequate solubility, stability and storage without significant loss of function. Importantly all, or substantially most, of the above properties should be achieved without a loss of the conditional targeting of the effector function and retention of conditional engagement of a modified signaling agent with its receptor. Often, it is difficult to achieve all these objectives with chimeric proteins. There is a need in the art where such desirable therapeutic properties of the biologic can be achieved while maintaining the tolerability and therapeutic index of the biologic, as well as ensuring adequate solubility, stability and storage of the pharmaceutical composition.

### SUMMARY

Accordingly, in aspects, the present application provides a pharmaceutical composition or formulation comprising (a) a C-type lectin domain family 9 member A (Clec9A)-based chimeric protein or chimeric protein complex (e.g., at a concentration of at least about 1 mg/mL) comprising (i) a targeting moiety that specifically binds to Clec9A, (ii) a modified human IFNα2, and (iii) a modified Fc domain; (b) at least one pharmaceutically acceptable buffering agent comprising a histidine buffer (e.g., at a concentration of at least about 10 mM to no more than about 25 mM); and (c) at least one pharmaceutically acceptable excipient comprising a surfactant (e.g., polysorbate 20 (PS20) or poloxamer 188 (Pol188)), and wherein the pharmaceutical composition has a pH that ranges from at least about 4.0 to at least about 6.0.

The present technology provides, in part, pharmaceutical compositions and/or formulations including chimeric protein complexes that comprise biological therapeutic agents whose effector function can be delivered in a highly precise fashion to a target of choice, with limited or no cross-reactivities, and with limited or no systemic adverse events, while also providing improved pharmaceutical features, such as solubility, stability, and storage properties.

In embodiments, the pharmaceutical compositions or formulations are suitable for parenteral administration, such as intravenous administration (e.g., intravenous infusion or bolus injection).

In embodiments, the pharmaceutical compositions or formulations of the present application comprise one or more additional excipients selected from salts, amino acids, isotonicity agents, tonicifiers, and antioxidants.

In embodiments, the Clec9A-based chimeric protein complex comprises a polypeptide having at least about 95% identity with any one of SEQ ID NOs: 1-4 and 43 or at least about 98% identity with any one of SEQ ID NOs: 1-4 and 43 or at least about 99% identity with any one of SEQ ID Nos: 1-4 and 43. In embodiments, the chimeric protein complex comprises a polypeptide of any one of SEQ ID NOs: 1-4 and 43, optionally with 0, or 1, or 2, or 3, or 4, or 5 mutations. In embodiments, the chimeric protein complex comprises a polypeptide of any one of SEQ ID NOs: 1-4 and 43.

In aspects, the present application relates to methods for treating or preventing a cancer, comprising administering an effective amount of the pharmaceutical composition as disclosed herein to a patient in need thereof.

### BRIEF DESCRIPTION OF THE DRAWINGS

FIG. 1 depicts turbidity measurements at 500 nm of Clec9A-based chimeric protein complex incubated in 15 different buffer types with increasing amounts of PEG 6000.

FIG. 2 depicts the fluorescence profile of 8 selected design of experiment (DOE) concepts from Table 3 in acetate buffer and with ethylenediaminetetraacetic acid (EDTA). A first and second melting point were observed respectively around 55 °C and 68 °C.

FIG. 3 shows regression coefficient plots for the Tm-max (top) and maximum fluorescence (bottom) of peak 1 in Acetate buffer (left), Histidine-Acetate buffer (middle) and Histidine buffer (right).

FIGS. 4A-E show volume distribution of Clec9A-based chimeric protein complex before (green and red) or after vortexing (blue and black) in concept buffer 1 (red and blue) or concept buffer 6 (green and black) in the absence of surfactant (top) or the presence of PS20 (0.01% or 0.05%) or the presence of poloxamer188 (0.01% or 0.05%).

FIGS. 5A-B display RP-HPLC chromatogram overlays of samples stored at 25 °C/60% RH; the arrow indicates slightly higher RRT 0.86 peaks. FIG. 5A depicts formulation buffer 1 (F001/F002), and FIG. 5B depicts formulation buffer 2 (F003/F004). Black: 1 mg/mL T3d, Blue: 10 mg/mL T3d, Green: 1 mg/mL T1w, Light blue: 10 mg/mL T1w, Pink: 1 mg/mL T2w, Brown: 10 mg/mL T2w. For FIG. 5A, at the arrow indicating the RRT 0.86 peaks, the curves from top to bottom are brown, pink, blue, light blue, green, and black. For FIG. 5B, at the arrow indicating the RRT 0.86 peaks, the curves from top to bottom are brown, pink, light blue, green, blue, and black.

FIGS. 6A-B show clEF electropherogram of Clec9A-based chimeric protein complex at start of the study (FIG. 6A, 10 mg/mL) and after 18 months storage at −20 °C (FIG. 6B, 1 and 10 mg/mL).

### DETAILED DESCRIPTION

The present disclosure provides, in part, pharmaceutical compositions and/or formulations comprising C-type lectin domain family 9 member A (Clec9A)-based chimeric proteins or chimeric protein complexes, which, without wishing to be bound by theory, can be stably formulated for efficacious biological effect and convenient dosing schedules, dosing volumes, and patient-friendly delivery devices.

The present application provides, in aspects, a pharmaceutical composition or formulation comprising (a) a C-type lectin domain family 9 member A (Clec9A)-based chimeric protein or chimeric protein complex comprising (i) a targeting moiety that specifically binds to Clec9A, (ii) a modified human IFNα2, and (iii) a modified Fc domain; (b) at least one pharmaceutically acceptable buffering agent comprising a histidine buffer; and (c) at least one pharmaceutically acceptable excipient comprising a surfactant, and wherein the pharmaceutical composition or formulation has a pH that ranges from at least 4.0 to at least about 6.0.

The present technology provides, in part, pharmaceutical compositions and/or formulations including chimeric protein complexes that comprise biological therapeutic agents whose effector function can be delivered in a highly precise fashion to a target of choice, with limited or no cross-reactivities, and with limited or no systemic adverse events, while also providing improved pharmaceutical features, such as solubility, stability, and storage properties. In embodiments, the pharmaceutical compositions and/or formulations of the present application can be produced in a large scale. In embodiments, the pharmaceutical compositions and/or formulations of the present application provide an in vivo half-life that ensures adequate time of exposure to the drug to elicit a therapeutically beneficial effect. In embodiments, the pharmaceutical compositions and/or formulations of the present application are sized to avoid rapid clearance or limited tissue penetrance and bio-distribution. In embodiments, the pharmaceutical compositions and/or formulations of the present application provide adequate solubility, stability and/or storage properties without significant loss of function.

### Clec9A-Based Chimeric Protein Complexes

In aspects, the present application provides pharmaceutical compositions or formulations comprising a chimeric protein complex, where the chimeric protein complex includes a targeting moiety that specifically binds to C-type lectin domain family 9 member A (Clec9A), a modified human IFNα2, and a modified Fc domain.

In embodiments, the chimeric protein complex includes a modified human interferon α2. In embodiments, the modified IFN-α2 agent has reduced affinity and/or activity for the IFN-α/β receptor (IFNAR), i.e., IFNAR1 and/or IFNAR2 chains. In embodiments, the modified IFN-α2 agent has substantially reduced or ablated affinity and/or activity for the IFN-α/β receptor (IFNAR), i.e., IFNAR1 and/or IFNAR2 chains. In embodiments, the modified human interferon α2, as disclosed herein, has an amino acid sequence having at least about 95% identity with of SEQ ID NOs: 9 or 10. In embodiments, the modified human IFNα2 has an amino acid sequence having at least about 98% identity or at least about 99% identity with of SEQ ID NOs: 9 or 10. In embodiments, the modified human IFNα2 has 1-3 mutations relative to the amino acid sequence of SEQ ID NOs: 9 or 10. In embodiments, the modified human IFNα2 comprises a R149A mutation with respect to SEQ ID NOs: 9 or 10. In embodiments, the modified human IFNα2 comprises a A145G mutation with respect to SEQ ID NOs: 9 or 10.

In embodiments, the R149A mutation, or the equivalent thereof, is present in the IFN-α2, relative to SEQ ID NO: 9 or 10.

In embodiments, the R149A mutation, or the equivalent thereof, is not present in the IFN-α2, relative to SEQ ID NO: 9 or 10, and instead, another mutation is present. For instance, this alternative mutation could be at one of positions R33, R144, A145, M148, and L153, or equivalents thereof, relative to SEQ ID NO: 9 or 10. In embodiments, the alternative mutation is one of R33A, R144A, R144I, R144L, R144S, R144T, R144Y, A145D, A145G, A145H, A145K, A145Y, M148A, and L153A, or equivalents thereof, relative to SEQ ID NO: 9 or 10. For clarity, in embodiments, any reference to R149A herein may be replaced with one of R33A, R144A, R144I, R144L, R144S, R144T, R144Y, A145D, A145G, A145H, A145K, A145Y, M148A and L153A, or equivalents thereof, relative to SEQ ID NO: 9 or 10. In embodiments, any reference to R149A herein may be replaced with A145G, or equivalents thereof, relative to SEQ ID NO: 9 or 10.

In embodiments, the targeting moiety of the chimeric protein complex disclosed herein comprises a recombinant heavy-chain-only antibody (VHH). In embodiments, the VHH has an amino acid sequence of at least about 95% identity with of one of SEQ ID NOs: 11 or 12. In embodiments, the VHH has an amino acid sequence of at least about 98% identity with of one of SEQ ID NOs: 11 or 12 or at least about 99% identity with of one of SEQ ID NOs: 11 or 12. In embodiments, the VHH has an amino acid sequence of any one of SEQ ID NOs: 11 and 12.

In embodiments, the chimeric protein complex disclosed herein comprises two targeting moieties. In embodiments, the chimeric protein complex disclosed herein comprises two identical targeting moieties.

In embodiments, the chimeric protein complex disclosed herein comprises two targeting moieties. In embodiments, the chimeric protein complex disclosed herein comprises two non-identical targeting moieties. For example, in embodiments, the chimeric protein complex disclosed herein comprises targeting moieties (e.g., without limitation, VHHs) against Clec9A and PD-L1.

In embodiments, the chimeric protein complex disclosed herein include at least one Fc domain. In embodiments, the chimeric protein complex includes a modified Fc domain where the modified Fc domain includes one or more of the following mutations: P329G, K322Q, K322A, P331G, or P331S relative to any of one of SEQ ID NO: 13-16. In embodiments, the modified Fc domain includes one or more of the following mutations: P329G, K322Q, K322A, P331G, or P331S relative to human IgG1 Fc.

In embodiments, the chimeric protein complex includes a modified Fc domain that has an amino acid sequence having at least about 90% identity with SEQ ID NO: 13-16. In embodiments, the modified Fc domain has an amino acid sequence having at least about 93% identity with SEQ ID NO: 13-16. In embodiments, the modified Fc domain has an amino acid sequence having at least about 95% identity with SEQ ID NO: 13-16.

In embodiments, the chimeric protein complex comprises a polypeptide having at least about 95% identity with any one of SEQ ID NOs: 1-4 and 43. In embodiments, the chimeric protein complex comprises a polypeptide having at least about 98% identity with any one of SEQ ID NOs: 1-4 and 43 or at least about 99% identity with any one of SEQ ID Nos: 1-4 and 43. In embodiments, the chimeric protein complex comprises a polypeptide of SEQ ID NOs: 1-4 and 43 wherein the sequence has less than 10 mutations as compared to the selected sequence. In embodiments, the chimeric protein complex comprises a polypeptide of SEQ ID NOs: 1-4 and 43 wherein the sequence has less than 5 mutations as compared to the selected sequence.

In embodiments, the chimeric protein complex comprises a polypeptide that has an amino acid sequence of SEQ ID NO: 1. This sequence includes a single domain antibody (VHH) against Clec9A (i.e., R1CHCL50(opt4)), a linker (i.e., 5*GGS), and a Fc hole Ridgway sequence with LALA-KQ mutation (i.e., Fc hole Ridgway (LALA-KQ), see Ridgway et al., Protein Engineering 1996; 9:617-621, which is incorporated by reference in its entirety). This construct of sequence of SEQ ID NO: 1 is denoted as follows: Variation 1 VHH-Fc R1CHCL50(opt4)-5*GGS-Fc hole Ridgway (LALA-KQ).

In embodiments, the chimeric protein complex comprises a polypeptide that has an amino acid sequence of SEQ ID NO: 2. This sequence includes a single domain antibody (VHH) against Clec9A (i.e., R1CHCL50(opt4)), a linker (i.e., 5*GGS), and a Fc hole Merchant sequence with LALA-KQ mutation (i.e., Fc hole Merchant (LALA-KQ), see Merchant et al., Nature Biotechnology 1998; 16:677-681, which is incorporated by reference in its entirety). This construct of SEQ ID NO: 2 is denoted as follows: VHH-Fc: R1CHCL50(opt4)-5*GGS-Fc hole Merchant (LALA-KQ).

In embodiments, the chimeric protein complex comprises a polypeptide that has an amino acid sequence of SEQ ID NO: 3. This sequence includes a single domain antibody (VHH) against Clec9A (i.e., 3LEC89(opt4)), alinker (i.e., 5*GGS), and a Fc hole Ridgway sequence with LALA-KQ mutation (i.e., Fc hole Ridgway (LALA-KQ), see Ridgway et al., Protein Engineering 1996; 9:617-621, which is incorporated by reference in its entirety). This construct of SEQ ID NO: 3 is denoted as follows: VHH-Fc: 3LEC89(opt4)-5*GGS-Fc hole Ridgway (LALA-KQ)

In embodiments, the chimeric protein complex comprises a polypeptide that has an amino acid sequence of SEQ ID NO: 4. This sequence includes a single domain antibody (VHH) against Clec9A (i.e., 3LEC89(opt4)), alinker (i.e., 5*GGS), and a Fc hole Merchant sequence with LALA-KQ mutation (i.e., Fc hole Merchant (LALA-KQ), see Merchant et al., Nature Biotechnology 1998; 16:677-681, which is incorporated by reference in its entirety). This construct of SEQ ID NO: 4 is denoted as follows: VHH-Fc: 3LEC89(opt4)-5*GGS-Fc hole Merchant (LALA-KQ).

In embodiments, the chimeric protein complex comprises a polypeptide that has an amino acid sequence of SEQ ID NO: 43. This sequence includes a single domain antibody (VHH) against Clec9A (i.e., R1CHCL50(opt4)), a linker (i.e., 5*GGS), and a Fc hole Merchant sequence with LALA-KQ mutation and without C terminal lysine (i.e., Fc hole Merchant (LALA-KQ), see Merchant et al., Nature Biotechnology 1998; 16:677-681, which is incorporated by reference in its entirety).

The chimeric protein complex of the present application may further include an amino acid sequence having at least about 95% identity with any one of SEQ ID NOs: 5-8, 29-36, or 41-42. In embodiments, the chimeric protein complex comprises a polypeptide that has an amino acid sequence of at least about 98% identity with any one of SEQ ID NOs: 5-8, 29-36, or 41-42 or at least about 99% identity with any one of SEQ ID Nos: 5-8, 29-36, or 41-42. In embodiments, the chimeric protein complex comprises a polypeptide that has an amino acid sequence selected from SEQ ID NOs: 5-8, 29-36, or 41-42 wherein the sequence has less than 10 mutations as compared to the selected sequence. In embodiments, the chimeric protein complex comprises a polypeptide that has an amino acid sequence selected from SEQ ID NOs: 5-8, 29-36, or 41-42 wherein the sequence has less than 5 mutations as compared to the selected sequence.

In embodiments, the chimeric protein complex comprises a polypeptide that has an amino acid sequence of SEQ ID NO: 5. This sequence includes a modified human interferon α2b having a R149A mutations (i.e., huIFNa2B_R149A), a linker (i.e., 10*GGS-G), and a Fc knob Ridgway sequence with LALA-KQ mutation (i.e., Fc knob Ridgway (LALA-KQ), see Ridgway et al., Protein Engineering 1996; 9:617-621, which is incorporated by reference in its entirety). This construct of sequence of SEQ ID NO: 5 is denoted as follows: Variation 1 Fc-AFN: Fc knob Ridgway (LALA-KQ)-10*GGS-G-huIFNa2B_R149A.

In embodiments, the chimeric protein complex comprises a polypeptide that has an amino acid sequence of SEQ ID NO: 6. This sequence includes a modified human interferon α2b having R149A and T106E mutations (i.e., huIFNa2B_R149A_T106E), a linker (i.e., 10*GGS-G), and a Fc knob Ridgway sequence with LALA-KQ mutation (i.e., Fc knob Ridgway (LALA-KQ), see Ridgway et al., Protein Engineering 1996; 9:617-621, which is incorporated by reference in its entirety). This construct of sequence of SEQ ID NO: 6 is denoted as follows: Variation 2 Fc-AFN: Fc knob Ridgway (LALA-KQ)-10*GGS-G-huIFNa2B_R149A_T106E.

In embodiments, the chimeric protein complex comprises a polypeptide that has an amino acid sequence of SEQ ID NO: 7. This sequence includes a modified human interferon α2b having a R149A mutations (i.e., huIFNa2B_R149A), a linker (i.e., 10*GGS-G), and a Fc knob Merchant sequence with LALA-KQ mutation (i.e., Fc knob Merchant (LALA-KQ), see Merchant et al., Nature Biotechnology 1998; 16:677-681, which is incorporated by reference in its entirety). This construct of sequence of SEQ ID NO: 7 is denoted as follows: Variation 3 Fc-AFN: Fc knob Merchant (LALA-KQ)-10*GGS-G-huIFNa2B_R149A.

In embodiments, the chimeric protein complex comprises a polypeptide that has an amino acid sequence of SEQ ID NO: 8. This sequence includes a modified human interferon α2b having R149A and T106E mutations (i.e., huIFNa2B_R149A_T106E), a linker (i.e., 10*GGS-G), and a Fc knob Merchant sequence with LALA-KQ mutation (i.e., Fc knob Merchant (LALA-KQ), see Merchant et al., Nature Biotechnology 1998; 16:677-681, which is incorporated by reference in its entirety). This construct of sequence of SEQ ID NO: 8 is denoted as follows: Variation 4 Fc-AFN: Fc knob Merchant (LALA-KQ)-10*GGS-G-huIFNa2B_R149A_T106E.

In embodiments, the chimeric protein complex comprises a polypeptide that has an amino acid sequence of SEQ ID NO: 41. This sequence includes a modified interferon α2b having A145G mutation, a linker (i.e., 10*GGS-G), and a Fc knob Merchant sequence with LALA-KQ mutation (i.e., Fc knob Merchant (LALA-KQ), see Merchant et al., Nature Biotechnology 1998; 16:677-681, which is incorporated by reference in its entirety). This construct of sequence of SEQ ID NO: 41 is denoted as follows: Fc4′-IFNα2b_A145G.

In embodiments, the chimeric protein complex comprises a polypeptide that has an amino acid sequence of SEQ ID NO: 42. This sequence includes a modified interferon α2b having T106A and A145G mutations, a linker (i.e., 10*GGS-G), and a Fc knob Merchant sequence with LALA-KQ mutation (i.e., Fc knob Merchant (LALA-KQ), see Merchant et al., Nature Biotechnology 1998; 16:677-681, which is incorporated by reference in its entirety). This construct of sequence of SEQ ID NO: 42 is denoted as follows: Fc4′-IFNα2a_T106E_A145G.

In one embodiment, the chimeric protein complex includes (i) an amino acid sequence having at least about 95% identity with any one of SEQ ID NOs: 1 or 3 and (ii) an amino acid sequence having at least about 95% identity with any one of SEQ ID NOs: 5 or 6. In embodiments, the chimeric protein complex includes (i) an amino acid sequence having at least about 98% identity with any one of SEQ ID NOs: 1 or 3 and (ii) an amino acid sequence having at least about 98% identity with any one of SEQ ID NOs: 5 or 6. In embodiments, the chimeric protein complex includes (i) an amino acid sequence having at least about 99% identity with any one of SEQ ID NOs: 1 or 3 and (ii) an amino acid sequence having at least about 99% identity with any one of SEQ ID NOs: 5 or 6. In an embodiment, the chimeric protein complex includes (i) an amino acid sequence having at least about 95% identity with any one of SEQ ID NOs: 2 or 4 and (ii) an amino acid sequence having at least about 95% identity with any one of SEQ ID NOs: 7 or 8. In an embodiment, the chimeric protein complex includes (i) an amino acid sequence having at least about 98% identity with any one of SEQ ID NOs: 2 or 4 and (ii) an amino acid sequence having at least about 98% identity with any one of SEQ ID NOs: 7 or 8. In embodiments, the chimeric protein complex includes (i) an amino acid sequence having at least about 99% identity with any one of SEQ ID NOs: 2 or 4 and (ii) an amino acid sequence having at least about 99% identity with any one of SEQ ID NOs: 7 or 8.

In embodiments, the chimeric protein complex comprises (i) a polypeptide having an amino acid sequence having at least about 95% identity with SEQ ID NO: 2 and (ii) a polypeptide having an amino acid sequence having at least about 95% identity with any one of SEQ ID NOs: 31 or 32. In embodiments, the chimeric protein complex comprises (i) a polypeptide having an amino acid sequence having at least about 98% identity with SEQ ID NO: 2 and (ii) a polypeptide having an amino acid sequence having at least about 98% identity with any one of SEQ ID NOs: 31 or 32. In embodiments, the chimeric protein complex comprises (i) a polypeptide having an amino acid sequence having at least about 99% identity with SEQ ID NO: 2 and (ii) a polypeptide having an amino acid sequence having at least about 99% identity with any one of SEQ ID NOs: 31 or 32. In embodiments, the chimeric protein complex comprises (i) a polypeptide having an amino acid sequence having at least about 95% identity with SEQ ID NO: 43 and (ii) a polypeptide having an amino acid sequence having at least about 95% identity with any one of SEQ ID NOs: 41 or 42. In embodiments, the chimeric protein complex comprises (i) a polypeptide having an amino acid sequence having at least about 98% identity with SEQ ID NO: 43 and (ii) a polypeptide having an amino acid sequence having at least about 98% identity with any one of SEQ ID NOs: 41 or 42. In embodiments, the chimeric protein complex comprises (i) a polypeptide having an amino acid sequence having at least about 99% identity with SEQ ID NO: 43 and (ii) a polypeptide having an amino acid sequence having at least about 99% identity with any one of SEQ ID NOs: 41 or 42.

In embodiments, the chimeric protein complex comprises: (i) a polypeptide having an amino acid sequence having at least about 95% identity with any one of SEQ ID NOs: 1 or 3 and (ii) a polypeptide having an amino acid sequence having at least about 95% identity with any one of SEQ ID NOs: 5 or 6.

In embodiments, the chimeric protein complex comprises: (i) a polypeptide having an amino acid sequence having at least about 98% identity with any one of SEQ ID NOs: 1 or 3 and (ii) a polypeptide having an amino acid sequence having at least about 98% identity with any one of SEQ ID NOs: 5 or 6.

In embodiments, the chimeric protein complex comprises: (i) a polypeptide having an amino acid sequence having at least about 99% identity with any one of SEQ ID NOs: 1 or 3 and (ii) a polypeptide having an amino acid sequence having at least about 99% identity with any one of SEQ ID NOs: 5 or 6.

In embodiments, the chimeric protein complex comprises: (i) a polypeptide having an amino acid sequence having at least about 95% identity with any one of SEQ ID NOs: 2 or 4 and (ii) a polypeptide having an amino acid sequence having at least about 95% identity with any one of SEQ ID NOs: 7 or 8.

In embodiments, the chimeric protein complex comprises: (i) a polypeptide having an amino acid sequence having at least about 98% identity with any one of SEQ ID NOs: 2 or 4 and (ii) a polypeptide having an amino acid sequence having at least about 98% identity with any one of SEQ ID NOs: 7 or 8.

In embodiments, the chimeric protein complex comprises: (i) a polypeptide having an amino acid sequence having at least about 99% identity with any one of SEQ ID NOs: 2 or 4 and (ii) a polypeptide having an amino acid sequence having at least about 99% identity with any one of SEQ ID NOs: 7 or 8.

In embodiments, the chimeric protein complex comprises: (i) a polypeptide having an amino acid sequence having at least about 95% identity with SEQ ID NO: 2 and (ii) a polypeptide having an amino acid sequence having at least about 95% identity with any one of SEQ ID NOs: 31 or 32.

In embodiments, the chimeric protein complex comprises: (i) a polypeptide having an amino acid sequence having at least about 98% identity with SEQ ID NO: 2 and (ii) a polypeptide having an amino acid sequence having at least about 98% identity with any one of SEQ ID NOs: 31 or 32.

In embodiments, the chimeric protein complex comprises: (i) a polypeptide having an amino acid sequence having at least about 99% identity with SEQ ID NO: 2 and (ii) a polypeptide having an amino acid sequence having at least about 99% identity with any one of SEQ ID NOs: 31 or 32.

In embodiments, the chimeric protein complex comprises: (i) a polypeptide having an amino acid sequence having at least about 95% identity with SEQ ID NO: 43 and (ii) a polypeptide having an amino acid sequence having at least about 95% identity with any one of SEQ ID NOs: 41 or 42.

In embodiments, the chimeric protein complex comprises: (i) a polypeptide having an amino acid sequence having at least about 98% identity with SEQ ID NO: 43 and (ii) a polypeptide having an amino acid sequence having at least about 98% identity with any one of SEQ ID NOs: 41 or 42.

In embodiments, the chimeric protein complex comprises: (i) a polypeptide having an amino acid sequence having at least about 99% identity with SEQ ID NO: 43 and (ii) a polypeptide having an amino acid sequence having at least about 99% identity with any one of SEQ ID NOs: 41 or 42.

In embodiments, the present application is related to a multivalent or bivalent chimeric protein complex comprising: a polypeptide having a sequence at least about 95%, or at least about 97%, or at least about 98%, or at least about 99% identical to the amino acid sequence of SEQ ID NO: 17 and a polypeptide having a sequence at least about 95%, or at least about 97%, or at least about 98%, or at least about 99% identical to the amino acid sequence of SEQ ID NO: 7; a polypeptide having a sequence at least about 95%, or at least about 97%, or at least about 98%, or at least about 99% identical to the amino acid sequence of SEQ ID NO: 2 and a polypeptide having a sequence at least about 95%, or at least about 97%, or at least about 98%, or at least about 99% identical to the amino acid sequence of SEQ ID NO: 19; a polypeptide having a sequence at least about 95%, or at least about 97%, or at least about 98%, or at least about 99% identical to the amino acid sequence of SEQ ID NO: 18 and a polypeptide having a sequence at least about 95%, or at least about 97%, or at least about 98%, or at least about 99% identical to the amino acid sequence of SEQ ID NO: 7; a polypeptide having a sequence at least about 95%, or at least about 97%, or at least about 98%, or at least about 99% identical to the amino acid sequence of SEQ ID NO: 20 and a polypeptide having a sequence at least about 95%, or at least about 97%, or at least about 98%, or at least about 99% identical to the amino acid sequence of SEQ ID NO: 7; a polypeptide having a sequence at least about 95%, or at least about 97%, or at least about 98%, or at least about 99% identical to the amino acid sequence of SEQ ID NO: 2 and a polypeptide having a sequence at least about 95%, or at least about 97%, or at least about 98%, or at least about 99% identical to the amino acid sequence of SEQ ID NO: 22; or a polypeptide having a sequence at least about 95%, or at least about 97%, or at least about 98%, or at least about 99% identical to the amino acid sequence of SEQ ID NO: 21 and a polypeptide having a sequence at least about 95%, or at least about 97%, or at least about 98%, or at least about 99% identical to the amino acid sequence of SEQ ID NO: 7. In embodiments, the present application is related to a method for treating or preventing a cancer, comprising administering an effective amount of the multivalent or bivalent chimeric protein complex described herein to a patient in need thereof.

In embodiments, the present application is related to a chimeric protein complex comprising at least about two polypeptides having a sequence at least about 95%, or at least about 97%, or at least about 98%, or at least about 99%, or 100% identical to the amino acid sequence of any one of SEQ ID Nos: 1-43. In embodiments, the present application is related to a method for treating or preventing a cancer, comprising administering an effective amount of the chimeric protein complex comprising at least about two polypeptides having a sequence at least about 95%, or at least about 97%, or at least about 98%, or at least about 99%, or 100% identical to the amino acid sequence of any one of SEQ ID Nos: 1-43 to a patient in need thereof.

### Pharmaceutical Formulations and Dosage Forms

The formulation of Clec9A-based chimeric proteins and/or chimeric protein complexes, or variants thereof, may further comprise one or more pharmaceutically acceptable carriers or excipients. As one skilled in the art will recognize, the formulations can be in any suitable form appropriate for the desired use and route of administration.

In embodiments, the administration of the pharmaceutical formulation including Clec9A-based chimeric proteins and/or chimeric protein complexes (and/or additional therapeutic agents) is intravenous and/or parenteral. In embodiments, routes of administration include, for example: intradermal, intramuscular, intraperitoneal, intravenous, subcutaneous, epidural, sublingual, intracerebral, intravaginal, or transdermal.

A pharmaceutical formulation including Clec9A-based chimeric proteins and/or chimeric protein complexes (and/or additional therapeutic agents) as described herein can be administered by any convenient route, for example, by intravenous infusion or bolus injection.

In some dosage forms, the Clec9A-based chimeric proteins and/or chimeric protein complexes described herein are mixed with at least one inert, pharmaceutically acceptable excipient or carrier.

In embodiments, the pharmaceutical compositions or formulations comprising Clec9A-based chimeric proteins and/or chimeric protein complexes described herein are generally buffered solutions. In embodiments, a buffer is a chemical agent that is able to absorb a certain quantity of acid or base without undergoing a strong variation in pH. Illustrative buffering agents include histidine buffer, citrate buffer, phosphate buffer, acetate buffer, succinate buffer, and bicarbonate buffer. In embodiments, the buffering agent comprises L-Histidine or L-Histidine hydrochloride monohydrate. In embodiments, when using the L-Histidine or L-Histidine hydrochloride monohydrate buffering system, L-Histidine or L-Histidine hydrochloride monohydrate is present at a concentration of from about 1 nM to about 50 mM, or from about 5 mM to about 45 mM, or from about 10 mM to about 35 mM, or from about 15 mM to about 30 mM, or from about 20 mM to about 25 mM. In embodiments, L-Histidine or L-Histidine hydrochloride monohydrate is present at a concentration of about 25 mM.

In embodiments, the pharmaceutical compositions or formulations described herein comprise a buffering agent further comprising an acetate buffer. In embodiments, the acetate buffer comprises acetic acid. In embodiments, the pharmaceutical compositions or formulations described herein comprise a buffering agent comprising an acetate-histidine buffer. In embodiments, acetate-histidine buffer comprises L-histidine and acetic acid.

In embodiments, the buffer maintains the pH of the formulation in the range of about pH 4.0 to about pH 6.0, or about 5.0 to about 6.0, or about 4.5 to about 5.5. In embodiments, the pharmaceutical compositions or formulations described herein have a pH of about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, or about 6.0. In embodiments, the formulation is pH adjusted, e.g., with hydrochloric acid and/or sodium hydroxide.

In embodiments, the pharmaceutical compositions or formulations comprising Clec9A-based chimeric proteins and/or chimeric protein complexes described herein additionally include a surface active agent. In embodiments, surface active agents suitable for use include, but are not limited to, any pharmaceutically acceptable, non-toxic surfactant. In embodiments, the formulation comprises a surfactant, which can act as a solubilizing agent. Classes of surfactants suitable for use in the compositions of the present application include, but are not limited to polysorbate 20, polysorbate 40, polysorbate 60, polysorbate 80, poloxamer 188, polyethoxylated fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-olyoxypropylene block copolymers, sorbitan fatty acid esters, lower alcohol fatty acid esters, ionic surfactants, and mixtures thereof. In embodiments, pharmaceutical compositions or formulations described herein comprise one or more surfactants including, but not limited to, polysorbate 20 and poloxamer 188.

In embodiments, the pharmaceutical compositions or formulations comprising Clec9A-based chimeric proteins and/or chimeric protein complexes described herein comprise a surfactant (e.g. polysorbate 20 (PS20) or poloxamer 188), at a concentration of at least about 0.01% (w/v) to at least about 0.1% (w/v), or at least about 0.01% (w/v) to at least about 0.05% (w/v), or at least about 0.02% (w/v) to at least about 0.09% (w/v), or at least about 0.03% (w/v) to at least about 0.08% (w/v), or at least about 0.04% (w/v) to at least about 0.07% (w/v), or at least about 0.05% (w/v) to at least about 0.06% (w/v). In embodiments, the pharmaceutical compositions or formulations described herein comprise a surfactant at a concentration of about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), or about 0.1% (w/v).

In embodiments, the pharmaceutical compositions or formulations comprising Clec9A-based chimeric proteins and/or chimeric protein complexes described herein include one or more additional excipients selected from an isotonicity agent, an amino acid, and an antioxidant.

In embodiments, the amino acid excipient comprises one or more of arginine, glycine, glutamate, and histidine, optionally wherein the additional excipient is arginine. In embodiments, the pharmaceutical compositions or formulations described herein comprise arginine at a concentration of at least about 10 mM to no more than 50 mM. In embodiments, the pharmaceutical compositions or formulations described herein comprise arginine at a concentration of from about 10 mM to about 50 mM, or from about 20 mM to about 50 mM, or about 30 mM to about 50 mM, or about 40 mM to about 50 mM. In embodiments, arginine is present in the pharmaceutical composition or formulation at a concentration of about 10 mM, about 15 mM, about 20 mM, about 25 mM, about 30 mM, about 35 mM, about 40 mM, about 45 mM, about 50 mM, about 55 mM, about 60 mM, about 65 mM, or about 70 mM.

In embodiments, isotonicity agents, which can be used singly or in combination, include, but are not limited to, dextrose, sucrose, glycerin, trehalose, mannitol, sorbitol, arginine, sodium chloride, or potassium chloride. In embodiments, the pharmaceutical compositions or formulations described herein comprise one or more isotonicity agent excipients selected from sodium chloride, sucrose, mannitol, sorbitol, and trehalose.

In embodiments, the isotonicity agent comprises or consists of sodium chloride. In embodiments, the pharmaceutical compositions or formulations described herein comprise sodium chloride at a concentration of at least about 10 mM to no more than 50 mM. In embodiments, the pharmaceutical compositions or formulations described herein comprise sodium chloride at a concentration of from about 10 mM to about 50 mM, or from about 20 mM to about 50 mM, or about 30 mM to about 50 mM, or about 40 mM to about 50 mM. In embodiments, sodium chloride is present in the pharmaceutical composition or formulation at a concentration of about 10 mM, about 15 mM, about 20 mM, about 25 mM, about 30 mM, about 35 mM, about 40 mM, about 45 mM, about 50 mM, about 55 mM, about 60 mM, about 65 mM, or about 70 mM.

In embodiments, the isotonicity agent comprises or consists of a sugar, such as sucrose, mannitol, sorbitol, or trehalose. In embodiments, the pharmaceutical compositions or formulations described herein comprise a sugar at a concentration of at least about 50 mM to at least about 350 mM, or from at least about 100 mM to at least about 300 mM, or from at least about 150 mM to at least about 250 mM. In embodiments, a sugar isotonicity agent is present in the pharmaceutical composition or formulation at a concentration of about 50, mM, about 100 mM, about 150 mM, about 200 mM, about 250 mM, about 300 mM, or about 350 mM.

In embodiments, the pharmaceutical compositions or formulations comprising Clec9A-based chimeric proteins and/or chimeric protein complexes described herein further comprise an antioxidant excipient. In embodiments, the antioxidant excipient is selected from methionine and ethylenediaminetetraacetic acid (EDTA). In embodiments, the antioxidant is methionine. In embodiments, the pharmaceutical compositions or formulations described herein comprise an antioxidant at a concentration of at least about 1 mM to at least about 15 mM, or from at least about 1 mM to at least about 10 mM, or from at least about 2 mM to at least about 10 mM, or from at least about 3 mM to at least about 10 mM, or from at least about 1 mM to at least about 5 mM, or from at least about 2 mM to at least about 5 mM, or from at least about 3 mM to at least about 5 mM. In embodiments, an antioxidant excipient is present in the pharmaceutical composition or formulation at a concentration of about 1 mM, about 2 mM, about 3 mM, about 4 mM, about 5 mM, about 6 mM, about 7 mM, about 8 mM, about 9 mM, or about 10 mM.

In embodiments, pharmaceutical compositions or formulations described herein comprise a Clec9A-based chimeric protein or protein complex that is present at a concentration from at least about 0.25 mg/mL to at least about 15 mg/mL. In embodiments, pharmaceutical compositions or formulations described herein comprise a Clec9A-based chimeric protein or protein complex that is present at a concentration from at least about 0.25 mg/mL to at least about 10 mg/mL, from at least about 0.5 mg/mL to at least about 10 mg/mL, from at least about 0.75 mg/mL to at least about 10 mg/mL, from at least about 1 mg/mL to at least about 10 mg/mL, from at least about 1 mg/mL to at least about 15 mg/mL.

In embodiments, pharmaceutical compositions or formulations described herein comprise at least about 0.25 mg/mL, or at least about 0.5 mg/mL, or at least about 0.75 mg/mL, or at least about 1 mg/mL, or at least about 1.25 mg/mL, or at least about 1.5 mg/mL, or at least about 1.75 mg/mL, or at least about 2 mg/mL, at least about 3 mg/mL, at least about 4 mg/mL, at least about 5 mg/mL, at least about 6 mg/mL, or at least about 7 mg/mL, or at least about 8 mg/mL, or at least about 9 mg/mL, or at least about 10 mg/mL of a Clec9A-based chimeric protein or protein complex.

By way of illustration, in embodiments, the pharmaceutical compositions or formulations described herein comprise (a) a C-type lectin domain family 9 member A (Clec9A)-based chimeric protein complex comprising (i) a targeting moiety that specifically binds to Clec9A, (ii) a modified human IFNα2, and (iii) a modified Fc domain, wherein the Clec9A-based chimeric protein complex is present at a concentration from at least about 1 mg/mL to about 10 mg/mL; (b) a buffering agent comprising a histidine buffer, wherein histidine is present at a concentration of about 25 mM; and (c) an excipient comprising a surfactant selected from PS20 at a concentration of at least about 0.05% and Pol188 at a concentration ranging from at least about 0.01% to at least about 0.05%, wherein the pharmaceutical composition has a pH that ranges from at least about 4.5 to at least about 5.5.

In embodiments, the pharmaceutical compositions or formulations described herein comprise (a) a C-type lectin domain family 9 member A (Clec9A)-based chimeric protein complex comprising (i) a targeting moiety that specifically binds to Clec9A, (ii) a modified human IFNα2, and (iii) a modified Fc domain, wherein the Clec9A-based chimeric protein complex is present at a concentration of at least about 1 mg/mL; (b) a buffering agent comprising a histidine buffer, wherein histidine is present at a concentration ranging from about 10 mM to about 25 mM; (c) an excipient comprising a surfactant selected from PS20 at a concentration ranging from about 0.01% to about 0.1%, and Pol188 at a concentration ranging from about 0.01% to about 0.1%; (d) an amino acid excipient comprising arginine, wherein arginine is present at a concentration of at least about 10 mM to no more than about 50 mM; (e) an antioxidant excipient comprising methionine, wherein methionine is present at a concentration of at least about 1 mM to at least about 5 mM; and (f) an isotonicity agent comprising sodium chloride, wherein sodium chloride is present at a concentration of at least about 10 mM to no more than about 50 mM, and wherein the pharmaceutical composition has a pH that ranges from at least 4.5 to at least about 5.5.

In embodiments, the pharmaceutical compositions or formulations described herein comprise (a) a C-type lectin domain family 9 member A (Clec9A)-based chimeric protein complex comprising (i) a targeting moiety that specifically binds to Clec9A, (ii) a modified human IFNα2, and (iii) a modified Fc domain, wherein the Clec9A-based chimeric protein complex is present at a concentration from at least about 1 mg/mL to at least about 10 mg/mL; (b) a buffering agent comprising a histidine buffer, wherein histidine is present at a concentration of about 25 mM; (c) an excipient comprising a surfactant that is Pol188 at a concentration of about 0.05%; (d) an amino acid excipient comprising arginine at a concentration of about 50 mM; (e) an antioxidant excipient comprising methionine at a concentration of about 3 mM; and (f) an isotonicity agent comprising sodium chloride at a concentration of about 40 mM, and wherein the pharmaceutical composition has a pH of about 5.5.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. frozen and/or lyophilized compositions), which can be dissolved or suspended in sterile injectable medium immediately before use. They may contain, for example, suspending or dispersing agents known in the art.

In embodiments, pharmaceutical formulations including Clec9A-based chimeric proteins and/or chimeric protein complexes (and/or additional therapeutic agents) may be presented in unit dosage forms and may be prepared by any of the methods well known in the art of pharmacy. Such methods generally include the step of bringing the therapeutic agents into association with a carrier, which constitutes one or more accessory ingredients. In embodiments, the formulations are prepared by uniformly and intimately bringing the therapeutic agent into association with a liquid carrier, a finely divided solid carrier, or both, and then, if necessary, shaping the product into dosage forms of the desired formulation.

In embodiments, the pharmaceutical formulation comprises a preservative, such as, but not limited to, phenol, meta-cresol, or sodium benzoate.

Pharmaceutically acceptable carriers include, but are not limited to, water, saline, and glycerol. In embodiments, the pharmaceutical composition or formulation described herein comprises fixed oils, polyethylene glycol, propylene glycol or other solvents. In embodiments, the solvent is water.

In embodiments, the composition is contained in an injection device, such as a syringe or an injection pen. In embodiments, the composition is contained in an injection pen. Auto-injectors such as an “injection pen” are spring-loaded syringes designed to deliver a dose of a particular drug. By design, injection pens are easy to use and are intended for self-administration by patients, or administration by untrained personnel. Injection pens are designed to overcome the hesitation associated with self-administration of the needle-based drug delivery device. The injection pen keeps the needle tip shielded prior to injection and also has a passive safety mechanism to prevent accidental firing (injection). Injection depth can be adjustable or fixed and a function for needle shield removal may be incorporated. By pressing a button, the syringe needle is automatically inserted into the subcutaneous tissue and the drug is delivered. Once the injection is completed some injection pens have a visual or audible indication to confirm that the full dose has been delivered.

In embodiments, the injection device contains from 1 to 10 unit doses or from 1 to 5 unit doses. In embodiments, the unit doses are no more than about 1.5 mLs or about 1 mL in volume (whether or not contained or delivered by an injection device). In embodiments, the unit doses are no more than 0.8 mL in volume, or no more than 0.7 mL in volume. In embodiments, the injection device delivers a microdose, e.g., having a volume in the range of about 50 μL to about 500 μL, or a volume in the range of from about 75 μL to about 250 μL. In embodiments, the microdose has a volume of from 100 to 200 μL. The amount of active agent delivered per unit dose can be adjusted based on the desired frequency of administration.

In embodiments, the pharmaceutical composition or formulation of the present application is suitable for administration by subcutaneous, intramuscular, intradermal, or intravenous administration. In embodiments, the subject receives a unit dose of the pharmaceutical composition or formulation about once every 1 week, or about once every 2 weeks, or about once every 3 weeks, or about once every 4 weeks (e.g., about once per month), or about once every 6 weeks, or about once every 8 weeks (about once every 2 months).

In embodiments, the pharmaceutical composition or formulation described herein is freeze-dried to form a lyophilized powder. In aspects, the present application contemplates a kit comprising (a) the pharmaceutical composition or formulation of the present application that has been freeze-dried to form a lyophilized powder, (b) a vial (e.g., glass vial), and (c) an injection device (e.g., a syringe or injection pen).

In embodiments, the lyophilized formulation is marketed in pharmaceutical dosage form. The pharmaceutical dosage form of the present application, although in embodiments in the form of a vial, may be any suitable container, such as ampoules, syringes, co-vials, which are capable of maintaining a sterile environment. Such containers can be glass or plastic, provided that the material does not interact with the pharmaceutical formulation. In embodiments, the closure is a stopper, e.g., a sterile rubber stopper, or a siliconized stopper, which affords a seal. In embodiments, a vial will contain a lyophilized powder including about 0.5 to about 10 g/vial. Thus, in embodiments, there is provided a kit comprising a lyophilized powder of the Clec9A-based chimeric protein complex pharmaceutical formulation, a glass vial and optionally a syringe.

The lyophilized formulations of the present application may be reconstituted with water, such as Sterile Water for Injection, or other sterile fluid such as co-solvents, to provide an appropriate solution of Clec9A-based chimeric protein complex for administration, as through parenteral injection following further dilution into an appropriate intravenous admixture container, for example, including normal saline.

Liquid pharmaceutically administrable compositions can, for example, be prepared by dissolving, dispersing, etc. at least one chemical entity and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of chemical entities contained in such parenteral compositions is highly dependent on the specific nature thereof, as well as the activity of the chemical entities and the needs of the subject.

Preparation of the pharmaceutical composition or formulation may involve mixing, granulating and compressing or dissolving the ingredients as appropriate to the desired preparation. It will be appreciated that the form and character of the pharmaceutically acceptable excipient is dictated by the amount of active ingredient with which it is to be combined, the route of administration and other well-known variables. The excipient(s) must be “acceptable” in the sense of being compatible with the other ingredients of the formulation and not deleterious to the recipient thereof.

### Methods of Treatment

In aspects, the present application relates to a method for treating or preventing a cancer, comprising administering an effective amount of the pharmaceutical composition as disclosed herein to a patient in need thereof.

In embodiments, the present application relates to a method for treating or preventing a solid tumor. In embodiments, the present application relates to a method for treating or preventing a blood cancer.

The pharmaceutical composition can be used for the treatment or prevention of a cancer selected from one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

### DEFINITIONS

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

As used herein, the term “effective amount” refers to a quantity sufficient to achieve a desired therapeutic and/or prophylactic effect, e.g., an amount which results in the prevention of, or a decrease in a disease or disorder or one or more signs or symptoms associated with a disease or disorder. In the context of therapeutic or prophylactic applications, the amount of a composition administered to the subject will depend on the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compounds may be administered to a subject having one or more signs or symptoms of a disease or disorder. As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

Although the open-ended term “comprising,” as a synonym of terms such as including, containing, or having, is used herein to describe and claim the subject matter of the present application, or embodiments thereof, may alternatively be described using alternative terms such as “consisting of” or “consisting essentially of.”

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or pharmaceutical compositions for use and/or uses in the manufacture of a medicaments for treating the diseases or disorders described herein.

As used herein, Fc domain mutations are numbered according to EU convention (Edelman et al., PNAS 1969; 63 (1) 78-85, incorporated by reference in its entirety). As used herein, the term “LALA” mutation refers to a double mutant Fc domain having L234A mutation and a L235A mutation. As used herein, the term “KQ” mutation refers to a mutant Fc domain having a K322Q mutation.

Knob in hole mutants are those described in Ridgway et al., Protein Engineering 1996; 9:617-621, which is hereby incorporated by reference in its entirety, i.e. Y407T/T366Y.

Alternatively, knob in hole mutants are those described in Merchant et al., Nature Biotechnology 1998; 16:677-681, which is incorporated by reference in its entirety, i.e. S354C:T366W/Y349C:T366S:L368A:Y407V.

SEQUENCES

SEQ ID NO: 1 Variation 1 VHH-Fc R1CHCL50(opt4)-5*GGS-Fc hole Ridgway (LALA-KQ)
DVQLVESGGGLVQPGGSLRLSCAASGSFSSINVMGWYRQAPGKERELVARITNLGLPNYADSVKGRFTISRDNSKNT
VYLQMNSLRPEDTAVYYCYLVALKAEYWGQGTLVTVSSGGSGGSGGSGGSGGSDKTHTCPPCPAPEAAGGPSVFL
FPPKPKDTLMISRTPEVTCVVVDVSHEDPEVKFNWYVDGVEVHNAKTKPREEQYNSTYRVVSVLTVLHQDWLNGKE
YKCQVSNKALPAPIEKTISKAKGQPREPQVYTLPPSRDELTKNQVSLTCLVKGFYPSDIAVEWESNGQPENNYKTTPP
VLDSDGSFFLTSKLTVDKSRWQQGNVFSCSVMHEALHNHYTQKSLSLSPGK
SEQ ID NO: 2 Variation 2 VHH-Fc: R1CHCL50(opt4)-5*GGS-Fc hole Merchant (LALA-KQ)
DVQLVESGGGLVQPGGSLRLSCAASGSFSSINVMGWYRQAPGKERELVARITNLGLPNYADSVKGRFTISRDNSKNT
VYLQMNSLRPEDTAVYYCYLVALKAEYWGQGTLVTVSSGGSGGSGGSGGSGGSDKTHTCPPCPAPEAAGGPSVFL
FPPKPKDTLMISRTPEVTCVVVDVSHEDPEVKFNWYVDGVEVHNAKTKPREEQYNSTYRVVSVLTVLHQDWLNGKE
YKCQVSNKALPAPIEKTISKAKGQPREPQVCTLPPSRDELTKNQVSLSCAVKGFYPSDIAVEWESNGQPENNYKTTP
PVLDSDGSFFLVSKLTVDKSRWQQGNVFSCSVMHEALHNHYTQKSLSLSPGK
SEQ ID NO: 3 Variation 3 VHH-Fc: 3LEC89(opt4)-5*GGS-Fc hole Ridgway (LALA-KQ)
DVQLVESGGGLVQPGGSLRLSCAASGRIFSVNAMGWYRQAPGKQRELVAAITNQGAPTYADSVKGRFTISRDNSKN
TVYLQMNSLRPEDTAVYYCKAFTRGDDYWGQGTLVTVSSGGSGGSGGSGGSGGSDKTHTCPPCPAPEAAGGPSV
FLFPPKPKDTLMISRTPEVTCWVDVSHEDPEVKFNWYVDGVEVHNAKTKPREEQYNSTYRVVSVLTVLHQDWLNG
KEYKCQVSNKALPAPIEKTISKAKGQPREPQVYTLPPSRDELTKNQVSLTCLVKGFYPSDIAVEWESNGQPENNYKTT
PPVLDSDGSFFLTSKLTVDKSRWQQGNVFSCSVMHEALHNHYTQKSLSLSPGK
SEQ ID NO: 4 Variation 4 VHH-Fc: 3LEC89(opt4)-5*GGS-Fc hole Merchant (LALA-KQ)
DVQLVESGGGLVQPGGSLRLSCAASGRIFSVNAMGWYRQAPGKQRELVAAITNQGAPTYADSVKGRFTISRDNSKN
TVYLQMNSLRPEDTAVYYCKAFTRGDDYWGQGTLVTVSSGGSGGSGGSGGSGGSDKTHTCPPCPAPEAAGGPSV
FLFPPKPKDTLMISRTPEVTCVVVDVSHEDPEVKFNWYVDGVEVHNAKTKPREEQYNSTYRVVSVLTVLHQDWLNG
KEYKCQVSNKALPAPIEKTISKAKGQPREPQVCTLPPSRDELTKNQVSLSCAVKGFYPSDIAVEWESNGQPENNYKT
TPPVLDSDGSFFLVSKLTVDKSRWQQGNVFSCSVMHEALHNHYTQKSLSLSPGK
SEQ ID NO: 5 Variation 1 Fc-AFN: Fc knob Ridgway (LALA-KQ)-10*GGS-G-huIFNa2B_R149A
DKTHTCPPCPAPEAAGGPSVFLFPPKPKDTLMISRTPEVTCVVVDVSHEDPEVKFNWYVDGVEVHNAKTKPREEQY
NSTYRVVSVLTVLHQDWLNGKEYKCQVSNKALPAPIEKTISKAKGQPREPQVYTLPPSRDELTKNQVSLYCLVKGFYP
SDIAVEWESNGQPENNYKTTPPVLDSDGSFFLYSKLTVDKSRWQQGNVFSCSVMHEALHNHYTQKSLSLSPGKGGS
GGSGGSGGSGGSGGSGGSGGSGGSGGSGCDLPQTHSLGSRRTLMLLAQMRRISLFSCLKDRHDFGFPQEEFGNQ
FQKAETIPVLHEMIQQIFNLFSTKDSSAAWDETLLDKFYTELYQQLNDLEACVIQGVGVTETPLMKEDSILAVRKYFQRI
TLYLKEKKYSPCAWEVVRAEIMASFSLSTNLQESLRSKE
SEQ ID NO: 6 Variation 2 Fc-AFN: Fc knob Ridgway (LALA-KQ)-10*GGS-G-huIFNa2B_R149A_T106E
DKTHTCPPCPAPEAAGGPSVFLFPPKPKDTLMISRTPEVTCVVDVSHEDPEVKFNWYVDGVEVHNAKTKPREEQY
NSTYRWVSVLTVLHQDWLNGKEYKCQVSNKALPAPIEKTISKAKGQPREPQVYTLPPSRDELTKNQVSLYCLVKGFYP
SDIAVEWESNGQPENNYKTTPPVLDSDGSFFLYSKLTVDKSRWQQGNVFSCSVMHEALHNHYTQKSLSLSPGKGGS
GGSGGSGGSGGSGGSGGSGGSGGSGGSGCDLPQTHSLGSRRTLMLLAQMRRISLFSCLKDRHDFGFPQEEFGNQ
FQKAETIPVLHEMIQQIFNLFSTKDSSAAWDETLLDKFYTELYQQLNDLEACVIQGVGVEETPLMKEDSILAVRKYFQRI
TLYLKEKKYSPCAWEVVRAEIMASFSLSTNLQESLRSKE
SEQ ID NO: 7 Variation 3 Fc-AFN: Fc knob Merchant (LALA-KQ)-10*GGS-G-huIFNa2B_R149A
DKTHTCPPCPAPEAAGGPSVFLFPPKPKDTLMISRTPEVTCVVVDVSHEDPEVKFNWYVDGVEVHNAKTKPREEQY
NSTYRVVSVLTVLHQDWLNGKEYKCQVSNKALPAPIEKTISKAKGQPREPQVYTLPPCRDELTKNQVSLWCLVKGFY
PSDIAVEWESNGQPENNYKTTPPVLDSDGSFFLYSKLTVDKSRWQQGNVFSCSVMHEALHNHYTQKSLSLSPGKGG
SGGSGGSGGSGGSGGSGGSGGSGGSGGSGCDLPQTHSLGSRRTLMLLAQMRRISLFSCLKDRHDFGFPQEEFGN
QFQKAETIPVLHEMIQQIFNLFSTKDSSAAWDETLLDKFYTELYQQLNDLEACVIQGVGVTETPLMKEDSILAVRKYFQ
RITLYLKEKKYSPCAWEWVRAEIMASFSLSTNLQESLRSKE
SEQ ID NO: 8 Variation 4 Fc-AFN: Fc knob Merchant (LALA-KQ)-10*GGS-G-huIFNa2B_R149A_T106E
DKTHTCPPCPAPEAAGGPSVFLFPPKPKDTLMISRTPEVTCVVVDVSHEDPEVKFNWYVDGVEVHNAKTKPREEQY
NSTYRVVSVLTVLHQDWLNGKEYKCQVSNKALPAPIEKTISKAKGQPREPQVYTLPPCRDELTKNQVSLWCLVKGFY
PSDIAVEWESNGQPENNYKTTPPVLDSDGSFFLYSKLTVDKSRWQQGNVFSCSVMHEALHNHYTQKSLSLSPGKGG
SGGSGGSGGSGGSGGSGGSGGSGGSGGSGCDLPQTHSLGSRRTLMLLAQMRRISLFSCLKDRHDFGFPQEEFGN
QFQKAETIPVLHEMIQQIFNLFSTKDSSAAWDETLLDKFYTELYQQLNDLEACVIQGVGVEETPLMKEDSILAVRKYFQ
RITLYLKEKKYSPCAWEWVRAEIMASFSLSTNLQESLRSKE
SEQ ID NO: 9 Human IFNα2a (amino acid sequence)
CDLPQTHSLGSRRTLMLLAQMRKISLFSCLKDRHDFGFPQEEFGNQFQKAETIPVLHEMIQQIFNLFSTKDSSAAWDE
TLLDKFYTELYQQLNDLEACVIQGVGVTETPLMKEDSILAVRKYFQRITLYLKEKKYSPCAWEWVRAEIMRSFSLSTNL
QESLRSKE
SEQ ID NO: 10 Human IFNα2b (amino acid sequence)
CDLPQTHSLGSRRTLMLLAQMRRISLFSCLKDRHDFGFPQEEFGNQFQKAETIPVLHEMIQQIFNLFSTKDSSAAWDE
TLLDKFYTELYQQLNDLEACVIQGVGVTETPLMKEDSILAVRKYFQRITLYLKEKKYSPCAWEWRAEIMRSFSLSTNLQ
ESLRSKE
SEQ ID NO: 11 R1CHCL50_opt4 (anti-human Clec9a VHH)
DVQLVESGGGLVQPGGSLRLSCAASGSFSSINVMGWYRQAPGKERELVARITNLGLPNYADSVKGRFTISRDNSKNT
VYLQMNSLRPEDTAVYYCYLVALKAEYWGQGTLVTVSS
SEQ ID NO: 12 3LEC89_opt4 (anti-human Clec9a VHH)
DVQLVESGGGLVQPGGSLRLSCAASGRIFSVNAMGWYRQAPGKQRELVAAITNQGAPTYADSVKGRFTISRDNSKN
TVYLQMNSLRPEDTAVYYCKAFTRGDDYWGQGTLVTVSS
SEQ ID NO: 13 Amino acid sequence of the Fc (human IgG1)-with LALA mutations and Ridgway hole
DKTHTCPPCPAPEAAGGPSVFLFPPKPKDTLMISRTPEVTCWWVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKGFYP
SDIAVEWESNGQPENNYKTTPPVLDSDGSFFLTSKLTVDKSRWQQGNVFSCSVMHEALHNHYTQKSLSLSPGK
SEQ ID NO: 14 Amino acid sequence of the Fc (human IgG1)-with LALA mutations and Ridgway knob
DKTHTCPPCPAPEAAGGPSVFLFPPKPKDTLMISRTPEVTCVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLYCLVKGFYP
SDIAVEWESNGQPENNYKTTPPVLDSDGSFFLYSKLTVDKSRWQQGNVFSCSVMHEALHNHYTQKSLSLSPGK
SEQ ID NO: 15 Amino acid sequence of the Fc (human IgG1)-with LALA mutations and Merchant hole
DKTHTCPPCPAPEAAGGPSVFLFPPKPKDTLMISRTPEVTCVVDVSHEDPEVKFNWYVDGVEVHNAKTKPREEQY
NSTYRWSVLTVLHQDWLNGKEYKCKVSNKALPAPIEKTISKAKGQPREPQVCTLPPSRDELTKNQVSLSCAVKGFY
PSDIAVEWESNGQPENNYKTTPPVLDSDGSFFLVSKLTVDKSRWQQGNVFSCSVMHEALHNHYTQKSLSLSPGK
SEQ ID NO: 16 Amino acid sequence of the Fc (human IgG1)-with LALA mutations and Merchant knob
DKTHTCPPCPAPEAAGGPSVFLFPPKPKDTLMISRTPEVTCWWVDVSHEDPEVKFNWYVDGVEVHNAKTKPREEQY
NSTYRVVSVLTVLHQDWLNGKEYKCKVSNKALPAPIEKTISKAKGQPREPQVYTLPPCRDELTKNQVSLWCLVKGFY
PSDIAVEWESNGQPENNYKTTPPVLDSDGSFFLYSKLTVDKSRWQQGNVFSCSVMHEALHNHYTQKSLSLSPGK

Other sequences are identified elsewhere in the text.

### EXAMPLES

### Example 1: Preparation of Clec9A-Based Chimera Formulation Protein Bulk

The DHFR-deficient CHO DG44 cell line used as a host for the recombinant cell lines is an auxotroph for hypoxanthine and thymidine (HT). A Clec9A-based chimeric protein stable cell line was developed. A DHFR-deficient CHO DG44 clone, adapted to growth in suspension in chemically defined medium, was obtained and a parental cell bank was generated. CHO DG44 host cells were transfected by electroporation with linearized vectors encoding the Clec9A-based chimera:

R1CHCL50-opt4_5*GGS_Fc(huIgG1-LALA-KQ-KiH) and neomycin resistance.

R1CHCL50-opt4_5*GGS_Fc(huIgG1-LALA-KQ-KiH), underlined sequence represents the signal peptide, * terminal lysine clipped off during expression:

(SEQ ID NO: 44)
MEFGLSWLFLVAILKGVQCDVQLVESGGGLVQPGGSLRLSCAASGSFSSINVMGWYRQAPGKERELVARITNLGLPN
YADSVKGRFTISRDNSKNTVYLQMNSLRPEDTAVYYCYLVALKAEYWGQGTLVTVSSGGSGGSGGSGGSGGSDKT
HTCPPCPAPEAAGGPSVFLFPPKPKDTLMISRTPEVTCVVVDVSHEDPEVKFNWYVDGVEVHNAKTKPREEQYNST
YRWSVLTVLHQDWLNGKEYKCQVSNKALPAPIEKTISKAKGQPREPQVCTLPPSRDELTKNQVSLSCAVKGFYPSDI
AVEWESNGQPENNYKTTPPVLDSDGSFFLVSKLTVDKSRWQQGNVFSCSVMHEALHNHYTQKSLSLSPGK*.
Fc(huIgG1-LALA-KQ-KiH)_10*GGS_G_huIFNa2b(R149A) and DHFR.
Fc(huIgG1-LALA-KQ-KiH)_10*GGS_G_huIFNa2b(R149A), underlined sequence represents the signal
peptide:
(SEQ ID NO: 45)
MDMRVPAQLLGLLLLWLRGARCDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCQVSNKALPAPIEKTISKAKGQPREPQVYTL
PPCRDELTKNQVSLWCLVKGFYPSDIAVEWESNGQPENNYKTTPPVLDSDGSFFLYSKLTVDKSRWQQGNVFSCSV
MHEALHNHYTQKSLSLSPGKGGSGGSGGSGGSGGSGGSGGSGGSGGSGGSGCDLPQTHSLGSRRTLMLLAQMR
RISLFSCLKDRHDFGFPQEEFGNQFQKAETIPVLHEMIQQIFNLFSTKDSSAAWDETLLDKFYTELYQQLNDLEACVIQ
GVGVTETPLMKEDSILAVRKYFQRITLYLKEKKYSPCAWEWVRAEIMASFSLSTNLQESLRSKE.

Pools were selected under auxotrophic DHFR selection in CD-OptiCHO medium supplemented with 8 mM Glutamax. Three days after plating, the plates were subjected to increasing G418 concentration to a final concentration of 0.5 mg/mL and 500 nM methotrexate (MTX). Selected pools with the highest Clec9A-based chimeric protein expression were cloned by ClonePix processing. ClonePix clones with the highest Clec9A-based chimeric protein expression were subsequently subjected to a final cloning step by limiting dilution. Two clones were selected for larger scale production in a 1 L bioreactor run. The bioreactor was harvested by centrifugation and the supernatant purified by proteinA chromatography followed by cation-exchange chromatography on a SP Sepharose HP resin (Cytiva), after which a total of 100 mg of Clec9A-based chimeric protein was pooled from the top fractions.

### Example 2: Formulation Buffer Stabilizes Clec9A-Based Chimeric Protein at 10 mg/mL During Storage

The aim of this experiment was to screen buffers in order to identify formulations that ensure the stability of the Clec9A-based chimeric protein at a concentration of 10 mg/mL during storage. Specifically, 15 different buffers (different buffer type or pH) were prepared (see Table 1) for solubility screening of the Clec9A-based chimeric protein:
Citrate buffer pH 4.5, 5.0 and 6.0:
Histidine buffer pH 5.0, 5.5 and 6.5
Phosphate buffer pH 6.0, 7.0 and 7.5
Acetate buffer pH 4.5 and 5.5
Histidine—acetate buffer pH 5.0
Succinate buffer pH 5.5
Tris buffer pH 7.5
Phosphate—citrate buffer pH 6.5

TABLE 1
Composition per mL of the buffers for the Clec9A-
based chimeric protein solubility screening

| Buffer solution | | | Composition per mL | |
|---|---|---|---|---|
| | target | strength | | to weigh |
| Name | pH | (mM) | Buffer component | (mg) |
| Sodium citrate buffer | 4.5 | 200 | Citric acid monohydrate | 19.75 mg |
| | | | Sodium citrate dihydrate | 31.18 mg |
| Sodium citrate buffer | 5.0 | 200 | Citric acid monohydrate | 13.45 mg |
| | | | Sodium citrate dihydrate | 40.00 mg |
| Sodium citrate buffer | 6.0 | 200 | Citric acid monohydrate | 3.36 mg |
| | | | Sodium citrate dihydrate | 54.11 mg |
| Sodium Phosphate buffer | 6.0 | 200 | Disodium hydrogen phosphate dihydrate | 4.77 mg |
| | | | Sodium dihydrogen phosphate dihydrate | 27.02 mg |
| Sodium Phosphate buffer | 7.0 | 200 | Disodium hydrogen phosphate dihydrate | 21.63 mg |
| | | | Sodium dihydrogen phosphate dihydrate | 12.24 mg |
| Sodium Phosphate buffer | 7.5 | 200 | Disodium hydrogen phosphate dihydrate | 29.56 mg |
| | | | Sodium dihydrogen phosphate dihydrate | 5.29 mg |
| Sodium acetate buffer | 4.5 | 200 | Sodium acetate trihydrate | 10.53 mg |
| | | | Acetic acid | 7.03 μL |
| | | | | (7.37 mg) |
| Sodium acetate buffer | 5.5 | 200 | Sodium acetate trihydrate | 23.49 mg |
| | | | Acetic acid | 1.57 μL |
| | | | | (1.64 mg) |
| Histidine buffer | 5.0 | 200 | L-Histidine | 1.98 mg |
| | | | L-Histidine Hydrochloride monohydrate | 39.26 mg |
| Histidine buffer | 5.5 | 200 | L-Histidine | 5.86 mg |
| | | | L-Histidine Hydrochloride monohydrate | 34.00 mg |
| Histidine buffer | 6.5 | 200 | L-Histidine | 20.11 mg |
| | | | L-Histidine Hydrochloride monohydrate | 14.76 mg |
| Acetate - Histidine buffer | 5.0 | 200 | L-Histidine | 31.03 mg |
| | | | Acetic acid | 14 μL |
| | | | | (14.67 mg) |
| Succinate buffer | 5.5 | 200 | Succinic acid | 23.62 mg |
| | | | Sodium Hydroxide 5M | 65 μL |
| | | | | (13.00 mg) |
| Tris buffer | 7.5 | 200 | Tris base | 24.23 mg |
| | | | Hydrochloric acid 1M | 163 μL |
| | | | | (5.92 mg) |
| Citric - Sodium | 6.5 | 200 | Disodium hydrogen phosphate dihydrate | 30.76 mg |
| phosphate buffer | | | Citric acid monohydrate | 7.06 mg |

Solubility of the Clec9A-based chimeric protein was tested by adding the protein to increasing amounts of PEG 6000 in the 15 different buffers and by measuring turbidity as a read-out for protein aggregation. Specifically, buffer/PEG solutions were prepared in a 96 deep well plate: the 200 mM buffer stock solutions; H₂O, and the 50% (w/v) PEG6000 stock solution were mixed in a way that a fixed concentration of 55.6 mM buffer was present in each well and an increasing concentration (0%, 2.2%, 6.7%, 8.9%, 11.1%, 13.3%, 15.6%, 17.8%, 20.0%, 22.2%, 27.8%, and 33.3%) of PEG was reached within the same row.

The Clec9A-based chimeric protein solution was concentrated by dialysis (with a 6-8 kDa MWCO dialysis membrane) at 2-8 °C against 500 mL 10 mM Na-acetate, 100 mM NaCl, 10% PEG20000, pH 5.0. The sample was subsequently dialyzed twice against 500 mL 10 mM Na-acetate, 100 mM NaCl, pH 5.0 and filtered through a sterilizing 0.2 μm filter (Pall KM2EKVS). The final Clec9A-based chimeric protein stock solution had a concentration of 12.9 mg/mL.

For the precipitation assay, 90 μL of each well of the 96 deep well plate with buffer-PEG mixtures was transferred to an UV transparent 384 well plate. For every buffer, a separate row was used. Then, 10 μL of Clec9A-based chimeric protein was added to each buffer-PEG solution and mixing was performed by pipetting up and down. The final concentration was 50 mM for the buffer and 1.3 mg/mL for the Clec9A-based chimeric protein. Finally, turbidity measurement was performed with the plate reader. Absorbance was measured at 500 nm.

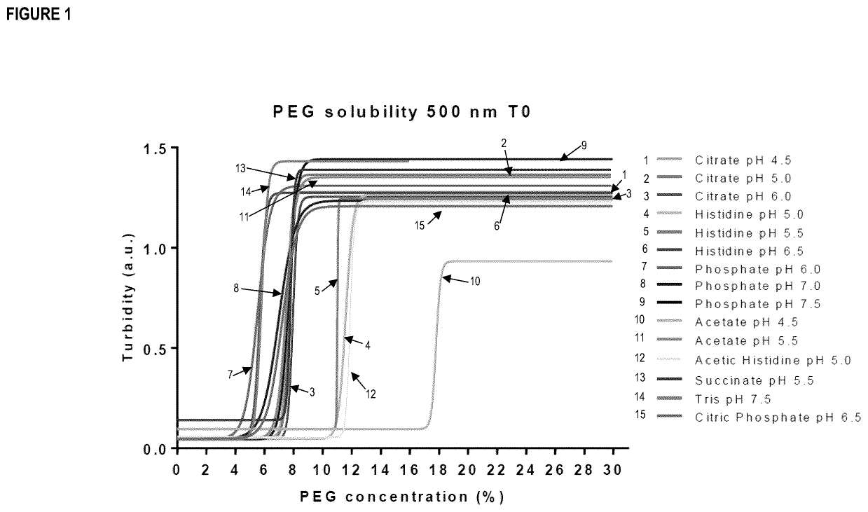

The turbidity measurements shown in FIG. 1 indicate that the best 4 buffers in which the Clec9A-based chimeric protein was less prone to precipitate were shown to be: acetate buffer at pH 4.5, acetic-histidine buffer at pH 5.0, histidine buffer at pH 5.0, and histidine buffer at pH 5.5. Surprisingly, citrate or succinate buffers in the same pH range were clearly less favorable for Clec9A-based chimeric protein solubility.

### Example 3: Screening of Formulation Excipients and Buffers for Stabilizing Influence

The Clec9A-based chimeric protein was concentrated by dialysis (with a 12-14 kDa MWCO dialysis membrane) at 2-8 °C against 2 L of 10 mM Na-acetate, 100 mM NaCl, 15% PEG20000, pH 4.5. The sample was subsequently dialyzed twice against 800 mL 10 mM Na-acetate, 100 mM NaCl, pH 4.5 (batch DVL-339-172B). The final Clec9A-based chimeric protein stock solution had a concentration of 14.8 mg/mL.

To cover the pH range 4.5 to 5.5, three (histidine buffer at pH 5.5, a histidine-acetate buffer at pH 5.0 and an acetate buffer at pH 4.5) of the four best buffers were selected for a design of experiment (DOE) to select candidate excipients. Although no (additional) oxidation of the Clec9A-based chimeric protein was observed after 24 hours incubation at room temperature (RT), the protein was found to be sensitive to H2O2-induced oxidation. Accordingly, antioxidants were also selected as excipients in a DOE with the following factors:
Factor 1: Buffer concentration
   Quantitative factor at 2 levels
   10 or 25 mM
Factor 2: Excipient 1 (Sugars)
   Qualitative factor at 5 levels
   Sucrose, Mannitol, Sorbitol, Trehalose or No sugar
   At a concentration of 200 mM
Factor 3: Excipient 2 (salts/amino acids)
   Qualitative factor at 4 levels
   Arginine, Glycine, NaCl or No extra salt/amino acids
   At a concentration of 50 mM
Factor 4: Excipient 3 (antioxidants)
   Qualitative factor at 3 levels
   EDTA, Methionine or no antioxidant
   At a concentration of 3 mM

Stock solutions were prepared at a concentration of 1000 mM for the sugars, 250 mM for the amino acids or the extra salt, 30 mM for the antioxidants and 500 mM for achieving the basic concentration of 50 mM NaCl as indicated in Table 2.

TABLE 2
Composition per mL of the buffers for Clec9A-based chimeric
protein or protein complex excipient screening

| Buffer solution | | | | | |
|---|---|---|---|---|---|
| | target | | Composition per mL | | |
| Name | pH | strength (mM) | Buffer component or excipient name | to weigh (mg) | |
| Sodium acetate buffer | 4.5 | 150 | Sodium acetate trihydrate | 7.90 mg | |
| | | | Acetic acid | 5.27 μL (5.52 mg) | |
| Acetate - Histidine buffer | 5.0 | 150 | L-Histidine | 10.48 mg | |
| | | | Acetic acid | 5 μL (5.24 mg) | |
| Histidine buffer | 5.5 | 150 | L-Histidine | 4.60 | mg |
| | | | L-Histidine Hydrochloride monohydrate | 24.80 | mg |
| Sucrose solution | — | 1000 | Sucrose | 342.30 | mg |
| Mannitol solution | — | 1000 | Mannitol | 182.2 | mg |
| Trehalose solution | — | 1000 | Trehalose dihydrate | 378.3 | mg |
| Sorbitol solution | — | 1000 | Sorbitol (liquid) | 201.6 μL | |
| | | | | (258.07 mg) | |
| Arginine | — | 250 | L-Arginine | 43.55 | mg |
| Glycine | — | 250 | Glycine | 18.78 | mg |
| NaCl (concentrated) | — | 500 | Sodium Chloride | 29.22 | mg |
| NaCl | — | 250 | Sodium Chloride | 14.61 | mg |
| Methionine | — | 30 | L-Methionine | 4.48 | mg |
| EDTA | — | 30 | Titriplex III | 11.17 | mg |

With these mainly qualitative factors, a design was created in MODDE (version 12.1, Sartorius). An amount of 93 combinations of the different factors were proposed. To investigate the validity of the model, 3 replicate measurements were added to the design resulting in a total number of 96 different concepts (Table 3). As this DOE design was performed once per buffer, a total of amount of 288 concepts were tested by thermal shift assay. To prepare the qPCR plates (Bioplastics), the concentrated stock solutions were mixed to achieve the final concentrations of 0.25 mg/mL of Clec9A-based chimeric protein or protein complex, 5× working concentration of Sypro Orange (stock solution is a 5000× from ThermoFisher), 50 mM NaCl, 10 or 25 mM buffer, 200 mM of sugar, 50 mM of amino acids or extra NaCl and 3 mM of antioxidant. A premixed solution of SYPRO Orange at a 30× working concentration and Clec9A-based chimeric protein or protein complex at a 1.5 mg/mL concentration was lastly added to the qPCR plate. A total volume of 30 μL was prepared. It was later observed that addition of arginine increased the pH of the solution substantially, though not at this point in the experiment, due to the lower sample volume.

TABLE 3
96 Excipient Concepts Tested Per Buffer
Condition in the Thermal Shift Assay

| No. | Buffer conc. (mM) | Excipient 1 | Excipient 2 | Excipient 3 |
|---|---|---|---|---|
| 1 | 10 | Sucrose | Arginine | EDTA |
| 2 | 25 | Sucrose | Arginine | EDTA |
| 3 | 10 | Mannitol | Arginine | EDTA |
| 4 | 25 | Mannitol | Arginine | EDTA |
| 5 | 10 | Sorbitol | Arginine | EDTA |
| 6 | 25 | Trehalose | Arginine | EDTA |
| 7 | 10 | NA | Arginine | EDTA |
| 8 | 25 | NA | Arginine | EDTA |
| 9 | 10 | Sucrose | NaCl | EDTA |
| 10 | 10 | Mannitol | NaCl | EDTA |
| 11 | 25 | Mannitol | NaCl | EDTA |
| 12 | 10 | Sorbitol | NaCl | EDTA |
| 13 | 25 | Sorbitol | NaCl | EDTA |
| 14 | 10 | Trehalose | NaCl | EDTA |
| 15 | 25 | Trehalose | NaCl | EDTA |
| 16 | 25 | NA | NaCl | EDTA |
| 17 | 25 | Sucrose | Glycine | EDTA |
| 18 | 25 | Mannitol | Glycine | EDTA |
| 19 | 10 | Sorbitol | Glycine | EDTA |
| 20 | 25 | Sorbitol | Glycine | EDTA |
| 21 | 10 | Trehalose | Glycine | EDTA |
| 22 | 25 | Trehalose | Glycine | EDTA |
| 23 | 10 | NA | Glycine | EDTA |
| 24 | 10 | Sucrose | NA | EDTA |
| 25 | 25 | Sucrose | NA | EDTA |
| 26 | 10 | Mannitol | NA | EDTA |
| 27 | 25 | Sorbitol | NA | EDTA |
| 28 | 10 | Trehalose | NA | EDTA |
| 29 | 25 | Trehalose | NA | EDTA |
| 30 | 10 | NA | NA | EDTA |
| 31 | 25 | NA | NA | EDTA |
| 32 | 10 | Sucrose | Arginine | Methionine |
| 33 | 25 | Sucrose | Arginine | Methionine |
| 34 | 10 | Mannitol | Arginine | Methionine |
| 35 | 25 | Mannitol | Arginine | Methionine |
| 36 | 25 | Sorbitol | Arginine | Methionine |
| 37 | 10 | Trehalose | Arginine | Methionine |
| 38 | 25 | Trehalose | Arginine | Methionine |
| 39 | 10 | NA | Arginine | Methionine |
| 40 | 25 | Sucrose | NaCl | Methionine |
| 41 | 25 | Mannitol | NaCl | Methionine |
| 42 | 10 | Sorbitol | NaCl | Methionine |
| 43 | 25 | Sorbitol | NaCl | Methionine |
| 44 | 10 | Trehalose | NaCl | Methionine |
| 45 | 10 | NA | NaCl | Methionine |
| 46 | 25 | NA | NaCl | Methionine |
| 47 | 10 | Sucrose | Glycine | Methionine |
| 48 | 25 | Sucrose | Glycine | Methionine |
| 49 | 10 | Mannitol | Glycine | Methionine |
| 50 | 10 | Sorbitol | Glycine | Methionine |
| 51 | 25 | Sorbitol | Glycine | Methionine |
| 52 | 10 | Trehalose | Glycine | Methionine |
| 53 | 25 | Trehalose | Glycine | Methionine |
| 54 | 25 | NA | Glycine | Methionine |
| 55 | 10 | Sucrose | NA | Methionine |
| 56 | 10 | Mannitol | NA | Methionine |
| 57 | 25 | Mannitol | NA | Methionine |
| 58 | 10 | Sorbitol | NA | Methionine |
| 59 | 25 | Sorbitol | NA | Methionine |
| 60 | 25 | Trehalose | NA | Methionine |
| 61 | 10 | NA | NA | Methionine |
| 62 | 25 | NA | NA | Methionine |
| 63 | 10 | Sucrose | Arginine | NA |
| 64 | 25 | Sucrose | Arginine | NA |
| 65 | 10 | Mannitol | Arginine | NA |
| 66 | 25 | Mannitol | Arginine | NA |
| 67 | 10 | Sorbitol | Arginine | NA |
| 68 | 25 | Sorbitol | Arginine | NA |
| 69 | 10 | Trehalose | Arginine | NA |
| 70 | 25 | NA | Arginine | NA |
| 71 | 10 | Sucrose | NaCl | NA |
| 72 | 25 | Sucrose | NaCl | NA |
| 73 | 10 | Mannitol | NaCl | NA |
| 74 | 10 | Sorbitol | NaCl | NA |
| 75 | 25 | Sorbitol | NaCl | NA |
| 76 | 25 | Trehalose | NaCl | NA |
| 77 | 10 | NA | NaCl | NA |
| 78 | 25 | NA | NaCl | NA |
| 79 | 10 | Sucrose | Glycine | NA |
| 80 | 10 | Mannitol | Glycine | NA |
| 81 | 25 | Mannitol | Glycine | NA |
| 82 | 25 | Sorbitol | Glycine | NA |
| 83 | 10 | Trehalose | Glycine | NA |
| 84 | 25 | Trehalose | Glycine | NA |
| 85 | 10 | NA | Glycine | NA |
| 86 | 25 | NA | Glycine | NA |
| 87 | 25 | Sucrose | NA | NA |
| 88 | 25 | Mannitol | NA | NA |
| 89 | 10 | Sorbitol | NA | NA |
| 90 | 10 | Trehalose | NA | NA |
| 91 | 25 | Trehalose | NA | NA |
| 92 | 10 | NA | NA | NA |
| 93 | 25 | NA | NA | NA |
| 94 | 25 | Trehalose | NA | NA |
| 95 | 25 | Trehalose | NA | NA |
| 96 | 25 | Trehalose | NA | NA |

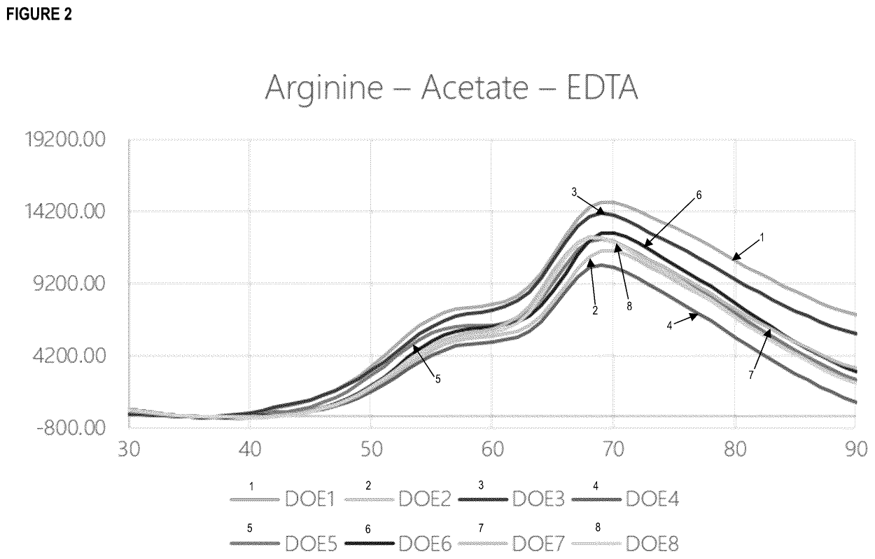

After incubation overnight at 2-8 °C, the fluorescence in function of temperature was measured with a qPCR of BioRad (CFX96 module on a C1000 PCR). The run was performed on a 96 well plate containing 30 μL of sample volume per well and the plate was sealed using optical seals (Bioplastics). A temperature ramp starting at 30 °C up to 90 °C (61 cycles) was programmed. The lid temperature was set at 105 °C. The HEX filter settings were used for excitation and emission. Remarkably, FIG. 2 shows that two melting points could be identified, which, without wishing to be bound by theory, may reflect different melting temperatures for the different domains (VHH, Fc and IFN moieties) in the complex molecule.

The analysis of the DOE was performed with MODDE software (version 12.1, Sartorius). Each set of responses (Tm-max and max. fluorescence) of the first peak (lowest melting point) was modelled and resulted in a good fit. The influence of each separate factor was visualized via a regression coefficient plot which visualizes how and to what extent the factor influences the response. A higher Tm-max value results in a more stable protein. The higher and more positive the coefficient in the regression plot, the higher the Tm-max and thus the more stabilization for the protein. For the maximum fluorescence, a lower maximum fluorescence is preferred as this reflects a lower amount of hydrophobic surface exposed to the environment and thus less denaturation and better stabilization. The more negative a coefficient, the more positive influence on the stabilization of the protein.

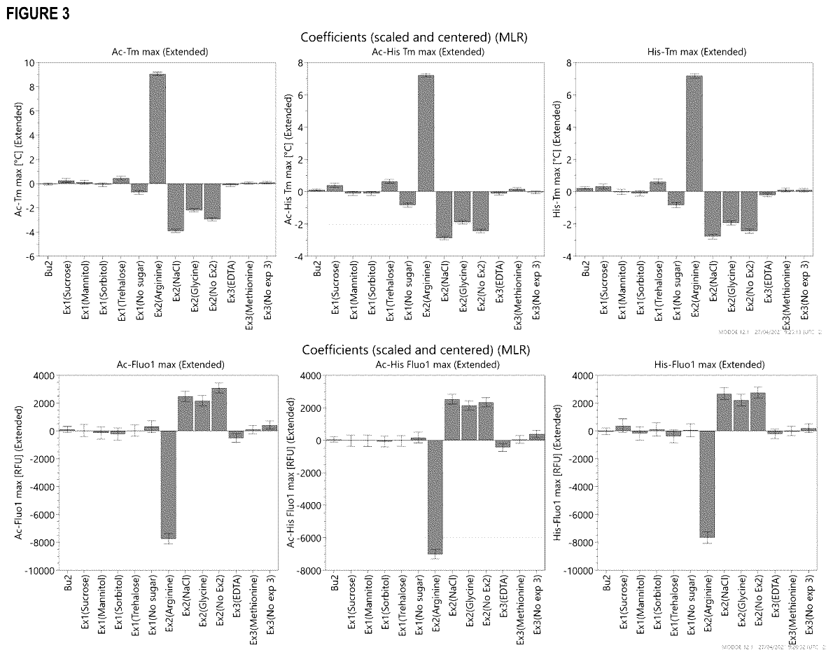

The regression coefficient plots for the Tm-max and the maximum fluorescence of peak 1 are depicted in FIG. 3 showing:
For both regression plots, arginine had a stabilizing influence in all buffers. As mentioned before, this can also be related to the increase in pH which was later observed for samples containing arginine. Although a higher pH is unlikely to contribute to stability (see solubility results in Examples 1), the role of arginine as a stabilizer was further evaluated in samples with and without arginine in the short stress study (see below).
Addition of glycine or extra NaCl and the absence of any extra salt or amino acid has a destabilizing influence.
The presence of sugars had, in general, no effect, with only trehalose showing slightly stabilizing effect on the Tm-max values in all buffers. Hence, sugars can be considered as an additional tonicifier.
Addition of antioxidants had only minor effects and hence both methionine or EDTA were evaluated in the short stress study (see below).

### Example 4: Short Stress Study

Based on the results of the buffer and excipient screening, five formulation buffers were tested for a short stress study, as depicted in Table 4. All concepts contained an antioxidant. Concept buffer 3 did not contain arginine in order to investigate the effect of this excipient without the pH shift that was observed before. The Clec9A-based chimeric protein or protein complex was dialyzed against the buffers of concept 1-3 with 15% (w/v) PEG20000 but without the antioxidant, which was spiked in after concentration to about 10 mg/mL. For concept buffer 4 and 5 remaining stock solution of the excipient screening was dialyzed against these buffers after which EDTA was spiked in and the concentration corrected to 10 mg/mL. The different concepts were stored at 40 °C/75% RH and at 2-8 °C for a maximum of two weeks. The samples were analyzed by RPC, SEC, and anti-IFN/anti-VHH binding ELISA.

TABLE 4
Overview of the different concepts for the short stress study

| Concept | Buffer type | | Arginine | Antioxidant | |
|---|---|---|---|---|---|
| Nr. | (25 mM) | pH | (mM) | (3 mM) | NaCl |
| 1 | Histidine | 5.5 | 50 | Methionine | 50 mM |
| 2 | Histidine | 5.5 | 50 | EDTA | 50 mM |
| 3 | Histidine | 5.5 | 0 | EDTA | 50 mM |
| 4 | Histidine-Acetate | 5.0 | 50 | EDTA | 50 mM |
| 5 | Acetate | 4.5 | 50 | EDTA | 50 mM |

For RPC, the Clec9A-based chimeric protein or protein complex samples were diluted to 1 mg/mL in 10 mM ammonium acetate, 123.5 mM NaCl at pH 5.5. Before HPLC analyses, samples were centrifuged at 20000 g for minutes, after which 2 μL was injected on UPLC C4 column (2.1 mm ID×100 mm) at 50 °C and proteins were eluted during a 15 min linear gradient ranging from 5 to 95% acetonitrile (with 0.1% TFA) at 0.35 mL/min flow rate. Proteins were detected by analytical flow cell (10 mm) at 214 and 280 nm wavelength. Table 5 depicts that the analysis indicated that for concepts 1 to 3 there was a significant amount of pre-peaks already formed immediately after formulation which were largely absent in concepts 4 and 5. The fraction of pre-peaks increased over time when the Clec9A-based chimeric protein or protein complex was stored at 40 °C, with concept 4 showing smaller increases as compared to concept 5. Of note, of the concepts with higher starting amounts of pre-peaks, concept 1 had the smallest increase.

TABLE 5
Results, as area % of each peak section, of short
stress study samples measured using RPC.

| | Concept 1 | Pre-peaks | Main peak | Post-peaks |
|---|---|---|---|---|
| 2-8 °C | T0 | 13.38 | 79.78 | 6.83 |
| | T1 w | 12.65 | 80.51 | 6.84 |
| | T2 w | 12.67 | 81.60 | 5.72 |
| 40 °C | T0 | 13.38 | 79.78 | 6.83 |
| | T1 w | 14.85 | 76.68 | 8.47 |
| | T2 w | 16.44 | 72.89 | 10.66 |
| | Concept 2 | Pre-peaks | Main peak | Post-peaks |
| 2-8 °C | T0 | 13.13 | 79.92 | 6.95 |
| | T1 w | 12.52 | 80.61 | 6.87 |
| | T2 w | 12.72 | 81.31 | 5.98 |
| 40 °C | T0 | 13.13 | 79.92 | 6.95 |
| | T1 w | 16.91 | 75.70 | 7.40 |
| | T2 w | 20.81 | 71.75 | 7.45 |
| | Concept 3 | Pre-peaks | Main peak | Post-peaks |
| 2-8 °C | T0 | 13.38 | 79.39 | 7.22 |
| | T1 w | 12.46 | 80.99 | 6.55 |
| | T2 w | 12.25 | 82.09 | 5.66 |
| 40 °C | T0 | 13.38 | 79.39 | 7.22 |
| | T1 w | 16.15 | 76.71 | 7.13 |
| | T2 w | 19.72 | 73.19 | 7.08 |
| | Concept 4 | Pre-peaks | Main peak | Post-peaks |
| 2-8 °C | T0 | 0.52 | 87.39 | 12.08 |
| | T1 w | 0.57 | 87.63 | 11.79 |
| | T2 w | 0.49 | 89.04 | 10.48 |
| 40 °C | T0 | 0.52 | 87.39 | 12.08 |
| | T1 w | 2.95 | 85.84 | 11.20 |
| | T2 w | 5.33 | 82.95 | 11.72 |
| | Concept 5 | Pre-peaks | Main peak | Post-peaks |
| 2-8 °C | T0 | 0.56 | 87.33 | 12.12 |
| | T1 w | 0.62 | 87.68 | 11.69 |
| | T2 w | 0.55 | 88.89 | 10.56 |
| 40 °C | T0 | 0.56 | 87.33 | 12.12 |
| | T1 w | 4.66 | 76.93 | 18.41 |
| | T2 w | 8.99 | 73.78 | 17.24 |

For SEC, the Clec9A-based chimeric protein or protein complex samples were diluted to 1 mg/mL in 10 mM ammonium acetate, 123.5 mM NaCl at pH 5.5. Before HPLC analyses, samples were centrifuged at 20000 g for 5 min and 10 μL was injected on Superdex 200 Increase SEC column (3.2 mm ID×300 mm) at room temperature and proteins were isocratically eluted by buffer identical to the dilution solvent at 0.075 mL/min flow rate. Proteins were detected by bio-compatible Titanium flow cell (5 mm) at 214 and 280 nm wavelength. The analysis, shown in Table 6, indicated that for concepts 1 to 3 there was a higher amount of pre-peaks already formed immediately after formulation, as compared to concepts 4 and 5. The fraction of pre-peaks increased over time when the Clec9A-based chimeric protein or protein complex was stored at 40 °C, with concept 4 showing smaller increases as compared to concept 5. Of note of the concepts with higher starting amounts of pre-peaks concept 1 had the smallest increase.

TABLE 6
Results, as area % of each peak section, of short
stress study samples measured using SEC.

| | Area % | | | | |
|---|---|---|---|---|---|
| | Concept | Pre- | | | Assay %* |
| | 1 | peaks | Main peak | Post-peaks | Main peak |
| 2-8 °C | T0 | 3.34 | 94.40 | 2.26 | 93.85 |
| | T1 w | 3.05 | 93.76 | 3.19 | 97.35 |
| | T2 w | 3.49 | 93.59 | 2.93 | 95.34 |
| 40 °C | T0 | 3.34 | 94.40 | 2.26 | 93.85 |
| | T1 w | 14.81 | 79.66 | 5.52 | 65.80 |
| | T2 w | 26.25 | 63.95 | 9.80 | 50.35 |
| | Concept | Pre- | | | |
| | 2 | peaks | Main peak | Post-peaks | Main peak |
| 2-8 °C | T0 | 3.65 | 94.10 | 2.25 | 95.48 |
| | T1 w | 3.10 | 93.93 | 2.98 | 95.50 |
| | T2 w | 3.55 | 93.77 | 2.67 | 95.39 |
| 40 °C | T0 | 3.65 | 94.10 | 2.25 | 95.48 |
| | T1 w | 23.91 | 70.44 | 5.65 | 61.78 |
| | T2 w | 43.05 | 50.47 | 6.48 | 43.78 |
| | Concept | Pre- | | | |
| | 3 | peaks | Main peak | Post-peaks | Main peak |
| 2-8 °C | T0 | 3.84 | 93.79 | 2.37 | 93.53 |
| | T1 w | 3.33 | 92.46 | 4.21 | 91.12 |
| | T2 w | 4.09 | 93.14 | 2.78 | 91.60 |
| 40 °C | T0 | 3.84 | 93.79 | 2.37 | 93.53 |
| | T1 w | 30.20 | 64.70 | 5.11 | 56.18 |
| | T2 w | 49.78 | 44.19 | 6.02 | 38.14 |
| | Concept | Pre- | | | |
| | 4 | peaks | Main peak | Post-peaks | Main peak |
| 2-8 °C | T0 | 0.38 | 99.11 | 0.52 | 94.59 |
| | T1 w | 0.51 | 98.97 | 0.52 | 96.55 |
| | T2 w | 0.68 | 98.98 | 0.34 | 97.16 |
| 40 °C | T0 | 0.38 | 99.11 | 0.52 | 94.59 |
| | T1 w | 34.68 | 63.63 | 1.70 | 50.90 |
| | T2 w | 46.58 | 49.33 | 4.10 | 29.75 |
| | Concept | Pre- | | | |
| | 5 | peaks | Main peak | Post-peaks | Main peak |
| 2-8 °C | T0 | 0.59 | 99.02 | 0.40 | 100.58 |
| | T1 w | 0.62 | 99.00 | 0.38 | 97.55 |
| | T2 w | 0.66 | 99.05 | 0.28 | 101.39 |
| 40 °C | T0 | 0.59 | 99.02 | 0.40 | 100.58 |
| | T1 w | 89.12 | 9.04 | 1.84 | 7.47 |
| | T2 w | 90.12 | 4.68 | 5.21 | 3.02 |

The anti-IFN/anti-VHH binding ELISA was performed to evaluate the correct folding of the VHH and/or IFN domains by recognition of specific antibodies. Briefly, MAXISORP Nunc immune plates were coated with anti-human IFN-alpha monoclonal antibody (clone MMHA-13; PBL assay science). After washing and blocking the plate, an 8-step 10-fold serial dilution in blocking buffer of Clec9A-based chimeric protein or protein complex samples starting from 10 mg/mL was loaded on the plate. After binding of Clec9A-based chimeric protein or protein complex to the plate, a first in-house made rabbit anti-VHH R1CHCL50 was used. A HRP-conjugated goat anti-rabbit (Jackson, 111-035-144, 1/20 000) antibody was used was a detection antibody. Detection was conducted using KLP substrate (Seracare) according to the manufacturer's instructions. The EC50 of the response curve was determined by 4PL analysis and expressed as relative value to a reference standard. The analysis indicated that at time point 0 and after storage at 2-8 °C the concepts 4 and 5 resulted in the best binding activity. Storage at 40 °C for 2 weeks resulted in a decrease of Clec9A-based chimeric protein or protein complex activity, especially when no histidine was added.

TABLE 7
Results, as % EC50 versus reference, of short
stress study samples measured using ELISA.

| Concept | T0 | T2 w 40 °C | T2 w 2-8 °C |
|---|---|---|---|
| Concept 1 | 70% | 46% | 89% |
| Concept 2 | 71% | 39% | 90% |
| Concept 3 | 75% | 33% | 82% |
| Concept 4 | 96% | 31% | 97% |
| Concept 5 | 86% | 6% | 95% |

In addition, all 5 concept formulations with Clec9A-based chimeric protein or protein complex at 10 mg/mL were subjected to four freeze/thaw cycles and analyzed by SEC and anti-IFN/anti-VHH binding ELISA. Overall, no impact was observed.

### Example 5: Shear Stress Stability

In order to investigate the influence of a surfactant on the formation of aggregation under shear stress, Clec9A-based chimeric protein or protein complex samples with different surfactants at different concentrations were stressed by vortexing. For these tests, two formulation buffers were tested as listed in Table 8. The surfactants Poloxamer 188 or PS20 were added from stock solutions to result in a final concentration of 0.01% or 0.05% (w/v).

TABLE 8
Overview of the different concepts tested
during the short stress study.

| Concept | Buffer type | | Arginine | Antioxidant | |
|---|---|---|---|---|---|
| Nr. | (25 mM) | pH | (mM) | (3 mM) | NaCI |
| 1 | Histidine | 5.5 | 50 | Methionine | 50 mM |
| 6 | Histidine-Acetate | 5.0 | 50 | Methionine | 50 mM |

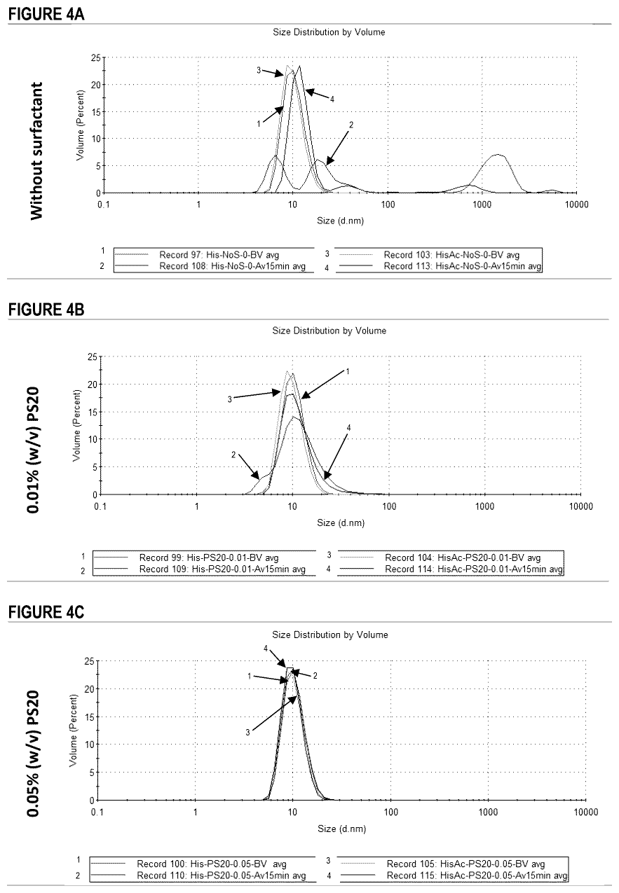

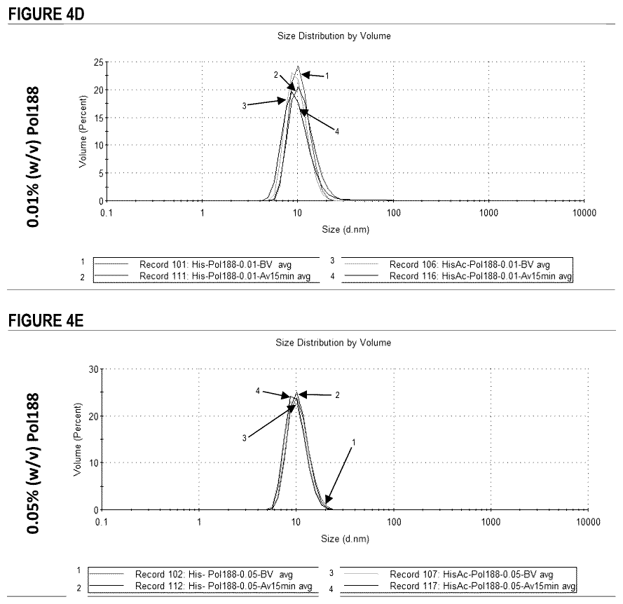

The impact of shear stress on the samples was followed with Dynamic Light Scattering (DLS). Samples are measured undiluted using a Malvern Zetasizer Nanoseries ZS at 25 °C with general purpose (normal resolution) analysis and non-invasive backscatter at 173 degrees as scattering mode. The hydrodynamic diameter of particles in the solution was measured before and after 15 minutes of vortexingError! Reference source not found. FIGS. 4A-E show the result of the particle sizes by volume distribution. Clec9A-based chimeric protein or protein complex is sensitive to shear stress in the absence of surfactants, but at a concentration of 0.05% (w/v) of surfactant, both surfactants offered sufficient protection against shear stress.

### Example 6: Accelerated Stability Studies of Two Selected Formulation Buffers

Two formulation buffers (see Table 9) were generated to follow accelerated stability testing of Clec9A-based chimeric protein or protein complex at 1 mg/mL and 10 mg/mL. Both buffers contained 0.05% (w/v) Poloxamer 188, which was added from a 5% (w/v) stock solution in the same buffer. 2R glass vials were filled with 2 mL of the Clec9A-based chimeric protein or protein complex formulations F001 (1 mg/mL of Clec9A-based chimeric protein or protein complex in histidine buffer), F002 (10 mg/mL of Clec9A-based chimeric protein or protein complex in histidine buffer), F003 (1 mg/mL of Clec9A-based chimeric protein or protein complex in histidine-acetate buffer) and F004 (10 mg/mL of Clec9A-based chimeric protein or protein complex in histidine-acetate buffer) and were closed with a bromobutyl stopper and aluminum cap. The samples were put in stability testing to be tested at −60 °C (1 month), 2-8 °C (2 weeks and 1 month), and 25 °C/60% RH (3 days, 1 week, and 2 weeks).

TABLE 9
Composition per mL of Two Selected Formulation Buffers for Accelerated Stability.

| | | | | Composition per mL | | |
|---|---|---|---|---|---|---|
| | Buffer solution | | | Buffer component or | | |
| | Name | target pH | strength (mM) | excipient | mg/μl | |
| BUFFER | Histidine-Arginine-NaCl- | 5.5 | 25 mM Histidine | L-Histidine | 3.88 | mg |
| 1 | Methionine buffer | | 50 mM Arg | L-Arginine monohydrochloride | 10.53 | mg |
| | | | 40 mM NaCl | Sodium chloride | 2.34 | mg |
| | | | 3 mM methionine | L-Methionine | 0.45 | mg |
| | 5% (w/V) Poloxamer 188 in | 5.5 | 0.05% (w/V) | 5% (w/v) Poloxamer 188 | 10.0 | μL |
| | Histidine-Arginine-NaCl- | | | | | |
| | Methionine buffer | | | | | |
| BUFFER | Histidine-Acetate-Arginine- | 5.0 | 25 mM Histidine- | L-Histidine | 1.75 | mg |
| 2 | NaCl-Methionine buffer | | acetate | Acetic acid | 0.83 | mg |
| | | | 50 mM Arg | L-Arginine monohydrochloride | 10.53 | mg |
| | | | 35 mM NaCl | Sodium chloride | 2.05 | mg |
| | | | 3 mM methionine | L-Methionine | 0.45 | mg |
| | 5% (w/V) Poloxamer 188 in | 5.0 | 0.05% (w/v) | Poloxamer 188 | 10.0 | μl |
| | Histidine-Acetate-Arginine- | | | | | |
| | NaCl-Methionine buffer | | | | | |

No changes in appearance (color and opalescence), osmolality, or pH of either formulation concept were observed during this study.

The area % data were acquired by SE-HPLC as previously described. There were no clear trends in the data, and all formulations appeared to be stable at these conditions.

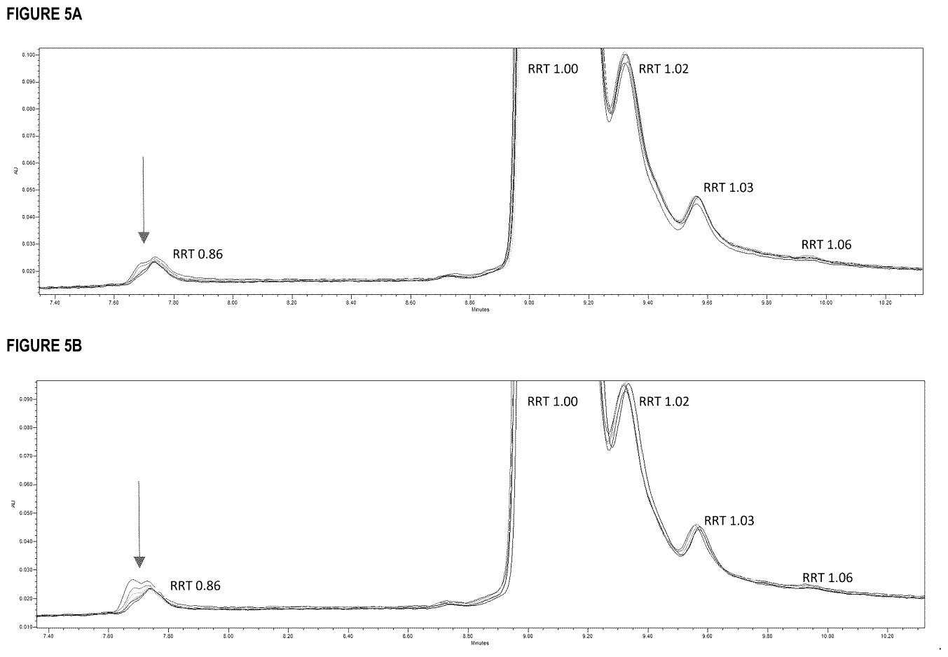

The area % data were acquired by RP-HPLC as previously described. FIGS. 5A-B display RP-HPLC chromatogram overlays of samples stored at 25 °C/60% RH; the arrow indicates slightly higher RRT 0.86 peaks. FIG. 5A depicts formulation buffer 1 (F001/F002), and FIG. 5B depicts formulation buffer 2 (F003/F004). Black: 1 mg/mL T3d, Blue: 10 mg/mL T3d, Green: 1 mg/mL T1w, Light blue: 10 mg/mL T1w, Pink: 1 mg/mL T2w, Brown: 10 mg/mL T2w. There was no clear trend exhibited in the <−60 and 2-8 °C data. Both formulation buffers at the two concentrations appeared stable at these conditions. At 25 °C/60% RH, there was a slight change at the pre-peak around RRT 0.86. This peak seemed to split into 2 peaks to a greater extent and/or during less storage time for concept 2 (F003/F004) than concept 1 (F001/F002), and for 10 mg/mL (F002/F004) than for 1 mg/mL (F001/F002). The total area of the pre-peak never exceeded 0.7%; thus, it was not possible to quantify the new peak which was only visually annotated by an arrow in FIGS. 5A-B.

The samples were also analyzed by the anti-VHH/anti-IFN ELISA as previously described. The results summarized in Table 10 indicate that, taking into account the normal biological variability for an ELISA, the binding activity of did not change over time for all conditions tested.

TABLE 10
Results, as % EC50 versus reference, of accelerated
stability study samples measured using ELISA.

| Storage | Formulation | T0 | T3 d | T1 W | T2 W | T1 M |
|---|---|---|---|---|---|---|
| 2-8 °C | F001 - 1 mg/mL | 99% | — | — | 116% | 99% |
| | F002 - 10 mg/mL | 105% | — | — | 103% | 98% |
| | F003 - 1 mg/mL | 97% | — | — | 99% | 90% |
| | F004 - 10 mg/mL | 106% | — | — | 103% | 94% |
| 25 °C/60% RH | F001 - 1 mg/mL | 99% | 105% | 118% | 100% | — |
| | F002 - 10 mg/mL | 105% | 103% | 114% | 99% | — |
| | F003 - 1 mg/mL | 97% | 107% | 111% | 109% | — |
| | F004 - 10 mg/mL | 106% | 112% | 106% | 104% | — |
| <−60 °C | F001 - 1 mg/mL | 99% | — | — | — | 93% |
| | F002 - 10 mg/mL | 105% | — | — | — | 96% |
| | F003 - 1 mg/mL | 97% | — | — | — | 96% |
| | F004 - 10 mg/mL | 106% | — | — | — | 93% |

In summary, after T1m, both tested formulation buffers, these being a Histidine buffer at pH 5.5 or a Histidine-Acetate buffer at pH 5.0, seemed to be able to stabilize Clec9A-based chimeric protein or protein complex up to 1 month when frozen or even when stored at 2-8 °C. Only at 25 °C/60% RH a minor difference in RPC chromatograms was observed with a minor new peak appearing slightly before RRT 0.86 for both buffers but which was (visually) somewhat higher for the Histidine-Acetate pH 5.0 buffer compared to the Histidine pH 5.5 buffer.

### Example 7: Long Term Clec9A-Based Chimeric Protein Complex Stability

Clec9A-based chimeric protein complex stability was evaluated for longer term in the histidine (25 mM)—arginine (50 mM)—Nacl (40 mM)—methionine (3 mM)—poloxamer 188 (0.05%, w/v) at pH 5.5 formulation buffer.

For this study, a new batch of Clec9A-based chimeric protein complex was produced in a 100 L bioreactor which was harvested by depth filtration and subsequently purified as before by proteinA and cation-exchange chromatography. In addition, a final polishing step was performed on the cation-exchange peak fractions using an anion-exchange filter (Polisher ST, 3M) after which the protein was concentrated by tangential flow filtration against the formulation buffer without the poloxamer. As a final step, the protein was diluted to 10 mg/mL and the poloxamer 188 added from a 5% stock concentration and aseptically filtered over a 0.22 μm filter. Part of the protein stock was subsequently compounded with formulation buffer to a 1 mg/mL Clec9A-based chimeric protein complex concentration.

Samples were stored at either 1 mg/mL or 10 mg/mL of Clec9A-based chimeric protein complex at −20 °C (1 month, 3 months, 6 months, 9 months, 12 months, 18 months, and 24 months), or at 2-8 °C (1 month, 3 months, 6 months).

Overall, no changes in appearance (color and opalescence) or pH at both concentration levels were observed during this study. Also, the Clec9A-based chimeric protein complex as measured by OD280 remained stable within the preset limits (10±1 mg/mL or 1.0±0.2 mg/mL).

SEC was performed as previously described. No additional high molecular weight species were formed during the entire study period; hence Table 11 only shows results for the starting material and the 6, 18 and 24 month time points.

TABLE 11
Results, as area % of each peak section, of long
term stability study samples as tested in SEC.

| | Concentration | | | | |
|---|---|---|---|---|---|
| | Clec9A-Based | | | | |
| | Chimeric Protein | | Results (% Area) | | |
| Time point | Complex | | HMW | Main peak | LMW |
| T0 | 1 | mg/mL | 0.3 | 99.3 | 0.4 |
| | 10 | mg/mL | 0.2 | 99.4 | 0.4 |
| T6 M (−20 °C) | 1 | mg/mL | 0.5 | 98.8 | 0.8 |
| | 10 | mg/mL | 0.9 | 98.3 | 0.8 |
| T6 M (2-8 °C) | 1 | mg/mL | 0.2 | 99.0 | 0.8 |
| | 10 | mg/mL | 0.6 | 98.5 | 0.9 |
| T18 M (−20 °C) | 1 | mg/mL | 0.2 | 98.5 | 1.3 |
| | 10 | mg/mL | 0.3 | 98.1 | 1.6 |
| T24 M (−20 °C) | 1 | mg/mL | 0.3 | 99.0 | 0.8 |
| | 10 | mg/mL | 0.2 | 99.0 | 0.8 |

RPC was performed as previously described. No changes in the Clec9A-based chimeric protein complex composition over time were observed during the entire study period; hence, Table 12 only shows results for the starting material and the 6-month, 18-month, and 24-month time points.

TABLE 12
Results, as area % of each peak section, of long
term stability study samples as tested in RPC.

| | Concentration | | | | | | |
|---|---|---|---|---|---|---|---|
| | Clec9A-Based | | Results (% Area) | | | | |
| | Chimeric Protein | | RRT | RRT | RRT | RRT | RRT |
| Time point | Complex | | 0.86 | 1.00 | 1.02 | 1.03 | 1.06 |
| T0 | 1 | mg/mL | 0.5 | 70.1 | 24.3 | 3.7 | 1.5 |
| | 10 | mg/mL | 0.5 | 70.3 | 24.5 | 3.7 | 1.1 |
| T6 M (−20 °C) | 1 | mg/mL | 0.5 | 68.4 | 24.4 | 4.4 | 2.3 |
| | 10 | mg/mL | 0.6 | 68.5 | 24.3 | 4.4 | 2.3 |
| T6 M (2-8 °C) | 1 | mg/mL | 0.6 | 67.8 | 24.4 | 4.6 | 2.6 |
| | 10 | mg/mL | 0.6 | 68.2 | 24.4 | 4.4 | 2.4 |
| T18 M (−20 °C) | 1 | mg/mL | 0.8 | 70.0 | 23.6 | 4.1 | 1.4 |
| | 10 | mg/mL | 0.8 | 69.5 | 23.5 | 4.1 | 2.0 |
| T24 M (−20 °C) | 1 | mg/mL | 0.7 | 70.2 | 23.4 | 3.6 | 1.9 |
| | 10 | mg/mL | 0.8 | 70.2 | 23.3 | 3.7 | 1.9 |

The samples were also analyzed by the anti-VHH/anti-IFN ELISA as previously described. Taking into account the normal biological variability for an ELISA, the binding activity of Clec9A-based chimeric protein complex did not change over time for all conditions tested; hence Table 13 only shows results for the starting material and the 6-month, 18-month, and 24-month time points.

TABLE 13
Results, as % EC50 versus reference, of accelerated
stability study samples measured using ELISA.

| Time point | Result 10 mg/mL | Result 1 mg/mL |
|---|---|---|
| T0 | 94% | 101% |
| T6 M (−20 °C) | 115% | 90% |
| T6 M (2-8 °C) | 124% | 92% |
| T18 M (−20 °C) | 115% | 109% |
| T24 M (−20 °C) | 93% | 104% |

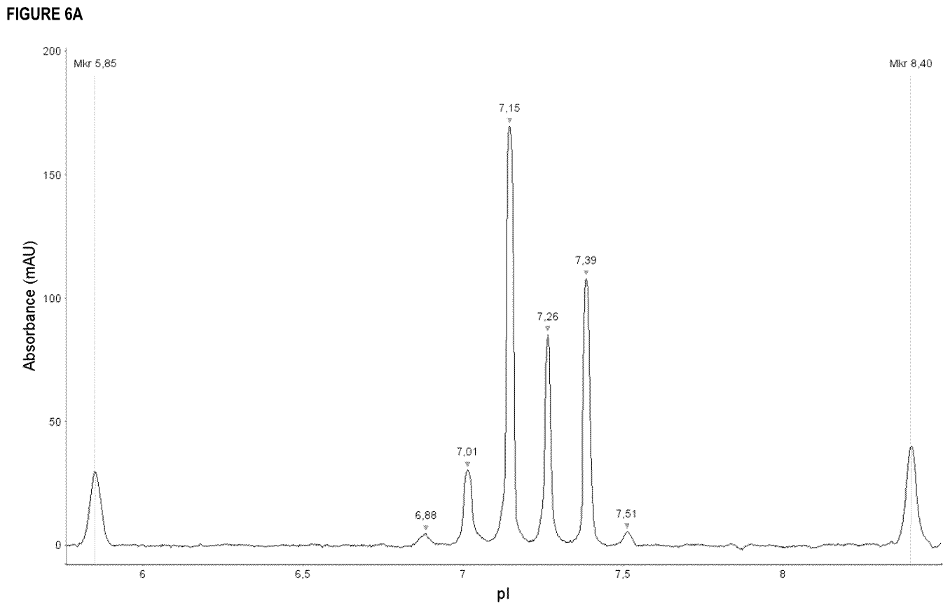

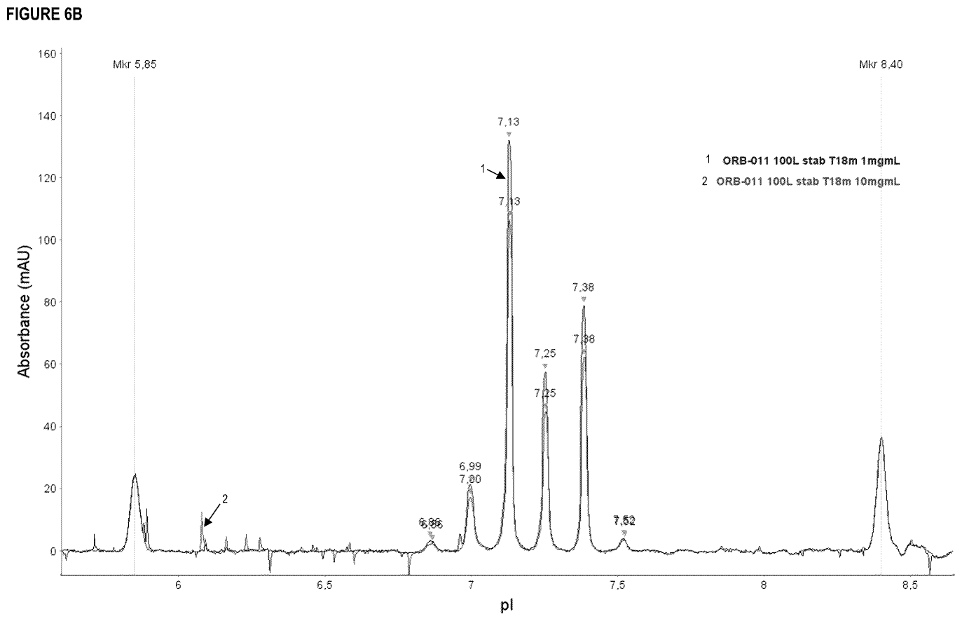

In addition, the samples were also tested for the appearance of new charge variants by capillary iso-electric focusing (clEF). In brief, the sample was diluted to a final concentration of 1 mg/mL. In a next step, this solution was desalted using a spin-column by loading 500 μL sample onto the column, centrifuging the column for 5 min at 14000×g, and bringing the volume back to 500 μL with MQ water. The desalting procedure was repeated two more times, after which 20 μL of the desalted sample was added to 80 μL of the clEF master mix (19.5 μL water, 19.5 μL 10 M urea, 34 μL 1% methyl cellulose, 1 μL Pharmalyte 3-10, 3 μL Pharmalyte 5-8, 2 μL 500 mM Arginine, 1 μL μl marker 5.85, 1 μL pl marker 8.40) into a microcentrifuge tube. The final concentration of Clec9A-based chimeric protein complex corresponded to 0.2 mg/mL. The samples were analyzed on the Maurice combo instrument (ProteinSimple) using a clEF cartridge PS-MC02-C (ProteinSimple). No new charge variants appeared over the entire study duration (see FIGS. 6A-B) and the relative amount of change variants remained unchanged over time, as observed during the entire study period (Table 14, only showing results for the starting material and the 6-month, 18-month, and 24-month time points).

TABLE 14
Results, as area % of each peak section, of long
term stability study samples as tested in cIEF.

| | Concentration | | | | | | | |
|---|---|---|---|---|---|---|---|---|
| | Clec9A-Based | | | | | | | |
| | Chimeric Protein | | Results (% Area) | | | | | |
| Time point | Complex | | pI 6.9 | pI 7.0 | pI 7.1 | pI 7.27 | pI 7.4 | pI 7.5 |
| T0 | 10 | m/mL | 1.5 | 9.7 | 41.4 | 19.7 | 26.0 | 1.6 |
| T6 M (−20 °C) | 1 | mg/mL | 1.4 | 9.5 | 41.3 | 19.9 | 26.2 | 1.7 |
| | 10 | mg/mL | 1.5 | 9.7 | 41.1 | 19.8 | 26.3 | 1.7 |
| T6 M (2-8 °C) | 1 | mg/mL | 1.6 | 9.9 | 41.1 | 19.9 | 25.9 | 1.7 |
| | 10 | mg/mL | 1.6 | 9.9 | 40.8 | 20.0 | 26.0 | 1.8 |
| T18 M (−20 °C) | 1 | mg/mL | 1.5 | 9.9 | 41.4 | 19.4 | 26.1 | 1.7 |
| | 10 | mg/mL | 1.3 | 9.7 | 41.3 | 19.7 | 26.2 | 1.8 |
| T24 M (−20 °C) | 1 | mg/mL | 1.2 | 10.2 | 41.7 | 19.3 | 25.9 | 1.6 |
| | 10 | mg/mL | 1.5 | 9.5 | 41.4 | 19.6 | 26.3 | 1.6 |

What is claimed is:

1. A pharmaceutical composition comprising:
(a) a C-type lectin domain family 9 member A (Clec9A)-based chimeric protein complex comprising (i) a targeting moiety that specifically binds to Clec9A, (ii) a modified human IFNα2, and (iii) a modified Fc domain;
(b) at least one pharmaceutically acceptable buffering agent comprising a histidine buffer; and
(c) at least one pharmaceutically acceptable excipient comprising a surfactant,
wherein the pharmaceutical composition has a pH that ranges from at least about 4.0 to at least about 6.0.

2. The pharmaceutical composition of claim 1, wherein the pH ranges from at least about 4.5 to at least about 5.5, optionally wherein the pH is about 5.5.

3. The pharmaceutical composition of claim 1 or claim 2, wherein the pharmaceutical composition is formulated for parenteral administration.

4. The pharmaceutical composition of claim 3, wherein the pharmaceutical composition is formulated for intravenous administration, optionally wherein intravenous administration comprises intravenous infusion or bolus injection.

5. The pharmaceutical composition of any one of the above claims, wherein the buffering agent is or comprises histidine at a concentration of at least about 10 mM to no more than about 25 mM.

6. The pharmaceutical composition of claim 5, wherein the buffering agent comprises histidine at a concentration of about 25 mM.

7. The pharmaceutical composition of any one of the above claims, wherein the buffering agent comprises L-Histidine or L-Histidine hydrochloride monohydrate.

8. The pharmaceutical composition of any one of the above claims, wherein the surfactant is or comprises a polysorbate surfactant, optionally selected from polysorbate 20 (PS20) and poloxamer 188 (Pol188).

9. The pharmaceutical composition of claim 8, wherein the PS20 is present at a concentration of at least about 0.01% (w/v) to at least about 0.1% (w/v), optionally wherein the PS20 is present at a concentration of about 0.05% (w/v).

10. The pharmaceutical composition of claim 8, wherein the Pol188 is present at a concentration of at least about 0.005% (w/v) to at least about 0.1% (w/v), optionally wherein the Pol188 is present at a concentration of at least about 0.01% (w/v) to at least about 0.05% (w/v).

11. The pharmaceutical composition of any one of the above claims, wherein the buffering agent further comprises an acetate buffer.

12. The pharmaceutical composition of claim 11, wherein the acetate buffer comprises acetic acid.

13. The pharmaceutical composition of any one of the above claims, further comprising one or more additional excipients selected from an amino acid, an isotonicity agent, and an antioxidant.

14. The pharmaceutical composition of claim 13, wherein the amino acid excipient is or comprises arginine.

15. The pharmaceutical composition of claim 14, wherein arginine is present at a concentration of at least about 10 mM to no more than about 50 mM, optionally wherein arginine is present at a concentration of about 50 mM.

16. The pharmaceutical composition of claim 13, wherein the isotonicity agent excipient is selected from sodium chloride, sucrose, mannitol, sorbitol, and trehalose.

17. The pharmaceutical composition of claim 16, wherein the isotonicity agent is sodium chloride, optionally wherein the sodium chloride is present at a concentration of at least about 10 mM to no more than about 50 mM, optionally wherein sodium chloride is present at a concentration of about 50 mM.

18. The pharmaceutical composition of claim 13, wherein the antioxidant excipient is selected from methionine and ethylenediaminetetraacetic acid (EDTA).

19. The pharmaceutical composition of claim 18, wherein the antioxidant is methionine, optionally wherein methionine is present at a concentration of at least about 1 mM to at least about 5 mM, optionally wherein methionine is present at a concentration of about 3 mM.

20. The pharmaceutical composition of any one of the above claims, wherein the Clec9A-based chimeric protein complex is present at a concentration of least about 1 mg/mL.

21. The pharmaceutical composition of any one of the above claims, wherein the Clec9A-based chimeric protein complex is present at a concentration from at least about 1 mg/mL to at least about 10 mg/mL.

22. The pharmaceutical composition of any one of the above claims, wherein the Clec9A-based chimeric protein complex is substantially in the form of a heterodimer.

23. The pharmaceutical composition of any one of the above claims, wherein the modified human IFNα2 has an amino acid sequence having at least about 95% identity with of SEQ ID NOs: 9 or 10.

24. The pharmaceutical composition of claim 23, wherein the modified human IFNα2 has an amino acid sequence having at least about 98% identity with of SEQ ID NOs: 9 or 10.

25. The pharmaceutical composition of claim 24, wherein the modified human IFNα2 has an amino acid sequence having at least about 99% identity with of SEQ ID NOs: 9 or 10.

26. The pharmaceutical composition of any one of the above claims, wherein the modified human IFNα2 has 1-3 mutations relative to the amino acid sequence of SEQ ID NOs: 9 or 10.

27. The pharmaceutical composition of claim 26, wherein the modified human IFNα2 comprises a R149A mutation with respect to SEQ ID NOs: 9 or 10 or one of a R33A, R144A, R144I, R144L, R144S, R144T, R144Y, A145D, A145G, A145H, A145K, A145Y, M148A and L153A mutation with respect to SEQ ID NOs: 9 or 10, optionally A145G.

28. The pharmaceutical composition of any one of the above claims, wherein the chimeric protein complex comprises a recombinant heavy-chain-only antibody (VHH).

29. The pharmaceutical composition of claim 28, wherein VHH has an amino acid sequence of at least about 95% identity with of one of SEQ ID NOs: 11 or 12.

30. The pharmaceutical composition of claim 29, wherein VHH has an amino acid sequence of at least about 98% identity with of one of SEQ ID NOs: 11 or 12.

31. The pharmaceutical composition of claim 30, wherein VHH has an amino acid sequence of at least about 99% identity with of one of SEQ ID NOs: 11 or 12.

32. The pharmaceutical composition of claim 31, wherein the VHH is has an amino acid sequence of any one of SEQ ID NOs: 11 and 12.

33. The pharmaceutical composition of any one of the above claims, wherein the modified Fc domain comprises one or more of the following mutations: P329G, K322Q, K322A, P331G, or P331S relative to any one of SEQ ID NO: 13-16.

34. The pharmaceutical composition of claim 33, wherein the modified Fc domain comprises one or more of the following mutations: P329G, K322Q, K322A, P331G, or P331S relative to human IgG1 Fc.

35. The pharmaceutical composition of claim 33 or 34, wherein the modified Fc domain has an amino acid sequence of at least about 90% identity with SEQ ID NO: 13-16.

36. The pharmaceutical composition of claim 33 or 34, wherein the modified Fc domain has an amino acid sequence of at least about 93% identity with SEQ ID NO: 13-16.

37. The pharmaceutical composition of claim 33 or 34, wherein the modified Fc domain has an amino acid sequence of at least about 95% identity with SEQ ID NO: 13-16.

38. The pharmaceutical composition of any one of the above claims, wherein the Clec9A-based chimeric protein complex comprises a polypeptide having an amino acid sequence having at least about 95%, or at least about 98%, or at least about 99% identity with any one of SEQ ID NOs: 1-4 and 43.

39. The pharmaceutical composition of any one of claims 1-37, wherein the Clec9A-based chimeric protein complex comprises a polypeptide having an amino acid sequence selected from SEQ ID NOs: 1-4 and 43 and less than 10 mutations to the amino acid sequence.

40. The pharmaceutical composition of claim 39, wherein the Clec9A-based chimeric protein complex comprises a polypeptide having an amino acid sequence selected from SEQ ID NOs: 1-4 and 43, and less than 5 mutations to the amino acid sequence.

41. The pharmaceutical composition of any one of claims 1-38, wherein the Clec9A-based chimeric protein complex comprises a polypeptide having an amino acid sequence selected from SEQ ID NOs: 1-4 and 43.

42. The pharmaceutical composition of any one of the above claims, wherein the Clec9A-based chimeric protein complex further comprises a polypeptide having an amino acid sequence having at least about 95% identity with any one of SEQ ID NOs: 5-8, 29-36, or 41-42.

43. The pharmaceutical composition of claim 42, wherein the Clec9A-based chimeric protein complex further comprises a polypeptide having an amino acid sequence having at least about 98% identity with any one of SEQ ID NOs: 5-8, 29-36, or 41-42.

44. The pharmaceutical composition of claim 43, wherein the Clec9A-based chimeric protein complex further comprises a polypeptide having an amino acid sequence having at least about 99% identity with any one of SEQ ID NOs: 5-8, 29-36, or 41-42.

45. The pharmaceutical composition of any one of claims 42-44, wherein the Clec9A-based chimeric protein complex comprises:
(i) a polypeptide having an amino acid sequence having at least about 95% identity with any one of SEQ ID NOs: 1 or 3 and
(ii) a polypeptide having an amino acid sequence having at least about 95% identity with any one of SEQ ID NOs: 5 or 6.

46. The pharmaceutical composition of claim 45, wherein the chimeric protein complex comprises:
(i) a polypeptide having an amino acid sequence having at least about 98% identity with any one of SEQ ID NOs: 1 or 3 and
(ii) a polypeptide having an amino acid sequence having at least about 98% identity with any one of SEQ ID NOs: 5 or 6.

47. The pharmaceutical composition of claim 46, wherein the chimeric protein complex comprises:
(i) a polypeptide having an amino acid sequence having at least about 99% identity with any one of SEQ ID NOs: 1 or 3 and
(ii) a polypeptide having an amino acid sequence having at least about 99% identity with any one of SEQ ID NOs: 5 or 6.

48. The pharmaceutical composition of any one of claims 42-44, wherein the chimeric protein complex comprises:
(i) a polypeptide having an amino acid sequence having at least about 95% identity with any one of SEQ ID NOs: 2 or 4 and
(ii) a polypeptide having an amino acid sequence having at least about 95% identity with any one of SEQ ID NOs: 7 or 8.

49. The pharmaceutical composition of claim 48, wherein the chimeric protein complex comprises:
(i) a polypeptide having an amino acid sequence having at least about 98% identity with any one of SEQ ID NOs: 2 or 4 and
(ii) a polypeptide having an amino acid sequence having at least about 98% identity with any one of SEQ ID NOs: 7 or 8.

50. The pharmaceutical composition of claim 49, wherein the chimeric protein complex comprises:
(i) a polypeptide having an amino acid sequence having at least about 99% identity with any one of SEQ ID NOs: 2 or 4 and
(ii) a polypeptide having an amino acid sequence having at least about 99% identity with any one of SEQ ID NOs: 7 or 8.

51. The pharmaceutical composition of any one of claims 42-44, wherein the chimeric protein complex comprises:
(i) a polypeptide having an amino acid sequence having at least about 95% identity with SEQ ID NO: 2 and
(ii) a polypeptide having an amino acid sequence having at least about 95% identity with any one of SEQ ID NOs: 31 or 32.

52. The pharmaceutical composition of claim 51, wherein the chimeric protein complex comprises:
(i) a polypeptide having an amino acid sequence having at least about 98% identity with SEQ ID NO: 2 and
(ii) a polypeptide having an amino acid sequence having at least about 98% identity with any one of SEQ ID NOs: 31 or 32.

53. The pharmaceutical composition of claim 52, wherein the chimeric protein complex comprises:
(i) a polypeptide having an amino acid sequence having at least about 99% identity with SEQ ID NO: 2 and
(ii) a polypeptide having an amino acid sequence having at least about 99% identity with any one of SEQ ID NOs: 31 or 32.

54. The pharmaceutical composition of any one of claims 42-44, wherein the chimeric protein complex comprises:
(i) a polypeptide having an amino acid sequence having at least about 95% identity with SEQ ID NO: 43 and
(ii) a polypeptide having an amino acid sequence having at least about 95% identity with any one of SEQ ID NOs: 41 or 42.

55. The pharmaceutical composition of claim 54, wherein the chimeric protein complex comprises:
(i) a polypeptide having an amino acid sequence having at least about 98% identity with SEQ ID NO: 43 and
(ii) a polypeptide having an amino acid sequence having at least about 98% identity with any one of SEQ ID NOs: 41 or 42.

56. The pharmaceutical composition of claim 55, wherein the chimeric protein complex comprises:
(i) a polypeptide having an amino acid sequence having at least about 99% identity with SEQ ID NO: 43 and
(ii) a polypeptide having an amino acid sequence having at least about 99% identity with any one of SEQ ID NOs: 41 or 42.

57. The pharmaceutical composition of any one of the above claims, wherein the pharmaceutical composition is freeze-dried.

58. The pharmaceutical composition of any one of the above claims, wherein the pharmaceutical composition forms a lyophilized powder.

59. The pharmaceutical composition of any one of the above claims, wherein the pharmaceutical composition is contained in an injection device, optionally wherein the injection device is a syringe or an injection pen.

60. A kit comprising the lyophilized powder of claim 58, a vial, and a syringe.

61. A method for treating or preventing a cancer, comprising administering an effective amount of the pharmaceutical composition of any one of claims 1-59 to a patient in need thereof.

62. The method of claim 61, wherein the cancer is a solid tumor.

63. The method of claim 61, wherein the cancer is a blood cancer.

64. The method of any one of claims 61-63, wherein the cancer is selected from one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

65. A pharmaceutical composition comprising:
(a) a C-type lectin domain family 9 member A (Clec9A)-based chimeric protein complex comprising (i) a targeting moiety that specifically binds to Clec9A, (ii) a modified human IFNα2, and (iii) a modified Fc domain, wherein the Clec9A-based chimeric protein complex is present at a concentration of at least about 1 mg/mL;
(b) a buffering agent comprising a histidine buffer, wherein histidine is present at a concentration ranging from about 10 mM to about 25 mM;
(c) an excipient comprising a surfactant selected from PS20 at a concentration ranging from about 0.01% to about 0.1%, and Pol188 at a concentration ranging from about 0.01% to about 0.1%;
(d) an amino acid excipient comprising arginine, wherein arginine is present at a concentration of at least about 10 mM to no more than about 50 mM;
(e) an antioxidant excipient comprising methionine, wherein methionine is present at a concentration of at least about 1 mM to at least about 5 mM; and
(f) an isotonicity agent comprising sodium chloride, wherein sodium chloride is present at a concentration of at least about 10 mM to no more than about 50 mM,
wherein the pharmaceutical composition has a pH that ranges from at least 4.5 to at least about 5.5.

66. A pharmaceutical composition comprising:
(a) a C-type lectin domain family 9 member A (Clec9A)-based chimeric protein complex comprising (i) a targeting moiety that specifically binds to Clec9A, (ii) a modified human IFNα2, and (iii) a modified Fc domain, wherein the Clec9A-based chimeric protein complex is present at a concentration from at least about 1 mg/mL to at least about 10 mg/mL;
(b) a buffering agent comprising a histidine buffer, wherein histidine is present at a concentration of about 25 mM;
(c) an excipient comprising a surfactant that is Pol188 at a concentration of about 0.05%;
(d) an amino acid excipient comprising arginine at a concentration of about 50 mM;
(e) an antioxidant excipient comprising methionine at a concentration of about 3 mM; and
(f) an isotonicity agent comprising sodium chloride at a concentration of about 40 mM,
wherein the pharmaceutical composition has a pH of about 5.5.
